(19) United States
(12) Patent Application Publication
(10) Pub. No.: US 2009/0192147 A1
(43) Pub. Date: Jul. 30, 2009

(54) [a]-FUSED INDOLE COMPOUNDS, THEIR USE AS mTOR KINASE AND PI3 KINASE INHIBITORS, AND THEIR SYNTHESES

(75) Inventors: Semiramis Ayral-Kaloustian, Tarrytown, NY (US); Nan Zhang, Bayside, NY (US); Aranapakam Mudumbai Venkatesan, Rego Park, NY (US); Tarek Suhayl Mansour, New City, NY (US); Thai Hiep Nguyen, Fair Lawn, NJ (US); James Thomas Anderson, Hillsdale, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/363,013

(22) Filed: Jan. 30, 2009

Related U.S. Application Data
(60) Provisional application No. 61/024,681, filed on Jan. 30, 2008.

(51) Int. Cl. A61K 31/5383; C07D 471/00; A61K 31/437; C07D 487/00; A61K 31/4985

(52) U.S. Cl. 514/230.2; 546/94; 514/294; 544/344; 514/250; 544/101

(57) ABSTRACT

The invention relates to [a]-fused indole compounds of the Formula II,

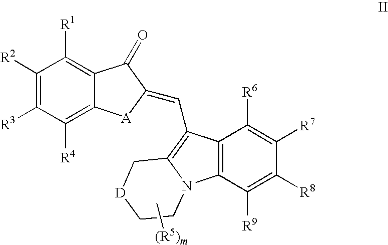

or a pharmaceutically acceptable salt thereof, wherein the constituent variables are as defined herein. The invention also relates to compositions comprising the compounds of Formula II, and methods for making and using the compounds.

### FIELD OF THE INVENTION

The invention relates to [a]-fused indole compounds, compositions comprising a [a]-fused indole compound, methods of synthesizing these compounds, and methods for treating PI3K-related diseases. The invention also relates to methods for treating mTOR-related diseases.

### BACKGROUND OF THE INVENTION

Phosphatidylinositol (hereinafter abbreviated as “PI”) is one of the phospholipids in cell membranes. In recent years it has become clear that PI plays an important role also in intracellular signal transduction. It is well recognized in the art that PI (4,5) bisphosphate (PI(4,5)P2 or PIP2) is degraded into diacylglycerol and inositol (1,4,5) triphosphate by phospholipase C to induce activation of protein kinase C and intracellular calcium mobilization, respectively [M. J. Berridge et al., Nature, 312, 315 (1984); Y. Nishizuka, Science, 225, 1365 (1984)].

In the late 1980s, phosphatidylinositol-3 kinase (“PI3K”) was found to be an enzyme that phosphorylates the 3-position of the inositol ring of phosphatidylinositol [D. Whitman et al., Nature, 332, 664 (1988)]. When PI3K was discovered, it was originally considered to be a single enzyme. Recently however, it was clarified that a plurality of PI3K subtypes exists. Three major subtypes of PI3Ks have now been identified on the basis of their in vitro substrate specificity, and these three are designated class I (a & b), class II, and class III [B. Vanhaesebroeck, Trend in Biol. Sci., 22, 267 (1997)].

The class Ia PI3K subtype has been most extensively investigated to date. Within the class Ia subtype there are three isoforms (α, β, & δ) that exist as hetero dimers of a catalytic 110-kDa subunit and regulatory subunits of 50-85 kDa. The regulatory subunits contain SH2 domains that bind to phosphorylated tyrosine residues within growth factor receptors or adaptor molecules and thereby localize PI3K to the inner cell membrane. At the inner cell membrane PI3K converts PIP2 to PIP3 (phosphatidylinositol-3,4,5-trisphosphate) that serves to localize the downstream effectors PDK1 and Akt to the inner cell membrane where Akt activation occurs. Activated Akt mediates a diverse array of effects including inhibition of apoptosis, cell cycle progression, response to insulin signaling, and cell proliferation. c Class Ia PI3K subtypes also contain Ras binding domains (RBD) that allow association with activated Ras providing another mechanism for PI3K membrane localization. Activated, oncogenic forms of growth factor receptors, Ras, and even PI3K kinase have been shown to aberrantly elevate signaling in the PI3K/Akt/mTOR pathway resulting in cell transformation. As a central component of the PI3K/Akt/mTOR signaling pathway PI3K (particularly the class Ia α isoform) has become a major therapeutic target in cancer drug discovery.

Substrates for class I PI3Ks are PI, PI(4)P and PI(4,5)P2, with PI(4,5)P2 being the most favored. Class I PI3Ks are further divided into two groups, class Ia and class Ib, because of their activation mechanism and associated regulatory subunits. The class Ib PI3K is p110γ that is activated by interaction with G protein-coupled receptors. Interaction between p110γ and G protein-coupled receptors is mediated by regulatory subunits of 110, 87, and 84 kDa.

PI and PI(4)P are the known substrates for class II PI3Ks; PI(4,5)P2 is not a substrate for the enzymes of this class. Class II PI3Ks include PI3K C2α, C2β and C2γ isoforms, which contain C2 domains at the C terminus, implying that their activity is regulated by calcium ions.

The substrate for class III PI3Ks is PI only. A mechanism for activation of the class III PI3Ks has not been clarified. Because each subtype has its own mechanism for regulating activity, it is likely that activation mechanism(s) depend on stimuli specific to each respective class of PI3K.

The compound P1103 (3-(4-(4-morpholinyl)pyrido[3′,2′:4,5]furo[3,2-d]pyrimidin-2-yl)phenol) inhibits PI3K_{α} and PI3K_{γ} as well as the mTOR enzymes with IC₅₀ values of 2, 3, and 50-80 nM respectively. I.P. dosing in mice of this compound in human tumor xenograft models of cancer demonstrated activity against a number of human tumor models, including the glioblastoma (PTEN null U87MG), prostate (PC3), breast (MDA-MB-468 and MDA-MB-435) colon carcinoma (HCT 116); and ovarian carcinoma (SKOV3 and IGROV-1); (Raynaud et al, Pharmacologic Characterization of a Potent Inhibitor of Class I Phosphatidylinositide 3-Kinases, Cancer Res. 2007 67: 5840-5850).

The compound ZSTK474 (2-(2-difluoromethylbenzoimidazol-1-yl)-4,6-dimorpholino-1, 3,5-triazine) inhibits PI3K_{α} and PI3K_{γ} but not the mTOR enzymes with an IC₅₀ values of 16, 4.6 and >10,000 nM respectively (Dexin Kong and Takao Yamori, ZSTK474 is an ATP-competitive inhibitor of class I phosphatidylinositol 3 kinase isoforms, Cancer Science, 2007, 98:10 1638-1642). Chronic oral administration of ZSTK474 in mouse human xenograft cancer models, completely inhibited growth which originated from a non-small-cell lung cancer (A549), a prostate cancer (PC-3), and a colon cancer (WiDr) at a dose of 400 mg/kg. (Yaguchi et al, Antitumor Activity of ZSTK474, a New Phosphatidylinositol 3-Kinase Inhibitor, J. Natl. Cancer Inst. 98: 545-556).

The compound NVP-BEZ-235 (2-methyl-2-(4-(3-methyl-2-oxo-8-(quinolin-3-yl)-2,3-dihydro-1H-imidazo[4,5-c]quinolin-1-yl)phenyl)propanenitrile) inhibits both PI3K_{α} and PI3K_{γ} as well as the mTOR enzymes with IC₅₀ values 4, 5, and “nanomolar”. Testing in human tumor xenograft models of cancer demonstrated activity against human tumor models of prostrate (PC-3) and glioblastoma (U-87) cancer. It entered clinical trials in December of 2006 (Verheijen, J. C. and Zask, A., Phosphatidylinositol 3-kinase (PI3K) inhibitors as anticancer drugs, Drugs Fut. 2007, 32(6): 537-547).

The compound SF-1126 (a prodrug form of LY-294002, which is 2-(4-morpholinyl)-8-phenyl-4H-1-benzopyran-4-one) is “a pan-PI3K inhibitor”. It is active in preclinical mouse cancer models of prostrate, breast, ovarian, lung, multiple myeloma, and brain cancers. It began clinical trials in April, 2007 for the solid tumors endometrial, renal cell, breast, hormone refractory prostate and ovarian cancers. (Verheijen, J. C. and Zask, A., Phosphatidylinositol 3-kinase (PI3K) inhibitors as anticancer drugs, Drugs Fut. 2007, 32(6): 537-547).

Exelixis Inc. (So. San Francisco, Calif.) recently filed INDs for XL-147 (a selective pan-PI3K inhibitor of unknown structure) and XL-765 (a mixed inhibitor of mTOR and PI3K of unknown structure) as anticancer agents. TargeGen's short-acting mixed inhibitor of PI3Kγ and δ, TG-100115, is in phase I/II trials for treatment of infarct following myocardial ischemia-reperfusion injury. Cerylid's antithrombotic PI3Kβ inhibitor CBL-1309 (structure unknown) has completed preclinical toxicology studies.

According to Verheijen, J. C. and Zask, A., Phosphatidylinositol 3-kinase (PI3K) inhibitors as anticancer drugs, Drugs Fut. 2007, 32(6): 537-547,

Although it seems clear that inhibition of the α isoform is essential for the antitumor activity of PI3K inhibitors, it is not clear whether a more selective inhibitor of a particular PI3K isoform may lead to fewer unwanted biological effects. It has recently been reported that non-PI3Kα class I isoforms (PI3Kβ, δ and γ) have the ability to induce oncogenic transformation of cells, suggesting that nonisoform-specific inhibitors may offer enhanced therapeutic potential over specific inhibitors.

Selectivity versus other related kinases is also an important consideration for the development of PI3K inhibitors. While selective inhibitors may be preferred in order to avoid unwanted side effects, there have been reports that inhibition of multiple targets in the PI3K/Akt pathway (e.g., PI3Kα and mTOR [mammalian target of rapamycin]) may lead to greater efficacy. It is possible that lipid kinase inhibitors may parallel protein kinase inhibitors in that nonselective inhibitors may also be brought forward to the clinic.

Mammalian Target of Rapamycin, mTOR, is a cell-signaling protein that regulates the response of tumor cells to nutrients and growth factors, as well as controlling tumor blood supply through effects on Vascular Endothelial Growth Factor, VEGF. Inhibitors of mTOR starve cancer cells and shrink tumors by inhibiting the effect of mTOR. All mTOR inhibitors bind to the mTOR kinase. This has at least two important effects. First, mTOR is a downstream mediator of the PI3K/Akt pathway. The PI3K/Akt pathway is thought to be over activated in numerous cancers and may account for the widespread response from various cancers to mTOR inhibitors. The over-activation of the upstream pathway would normally cause mTOR kinase to be over activated as well. However, in the presence of mTOR inhibitors, this process is blocked. The blocking effect prevents mTOR from signaling to downstream pathways that control cell growth. Over-activation of the PI3K/Akt kinase pathway is frequently associated with mutations in the PTEN gene, which is common in many cancers and may help predict what tumors will respond to mTOR inhibitors. The second major effect of mTOR inhibition is anti-angiogenesis, via the lowering of VEGF levels.

In lab tests, certain chemotherapy agents were found to be more effective in the presence of mTOR inhibitors. George, J. N., et al., Cancer Research, 61, 1527-1532, 2001. Additional lab results have shown that some rhabdomyosarcoma cells die in the presence of mTOR inhibitors. The complete functions of the mTOR kinase and the effects of mTOR inhibition are not completely understood.

There are three mTOR inhibitors, which have progressed into clinical trials. These compounds are Wyeth's Torisel, also known as 42-(3-hydroxy-2-(hydroxymethyl)-rapamycin 2-methylpropanoate, CCI-779 or Temsirolimus; Novartis' Everolimus, also known as 42-O-(2-hydroxyethyl)-rapamycin, or RAD 001; and Ariad's AP23573 also known as 42-(dimethylphopsinoyl)-rapamycin. The FDA has approved Torisel for the treatment of advanced renal cell carcinoma. In addition, Torisel is active in a NOS/SCID xenograft mouse model of acute lymphoblastic leukemia [Teachey et al, Blood, 107(3), 1149-1155, 2006]. Everolimus is in a phase II clinical study for patients with Stage IV Malignant Melanoma. AP23573 has been given orphan drug and fast-track status by the FDA for treatment of soft-tissue and bone sarcomas.

The three mTOR inhibitors have non-linear, although reproducible pharmacokinetic profiles. Mean area under the curve (AUC) values for these drugs increase at a less than dose related way. The three compounds are all semi-synthetic derivatives of the natural macrolide antibiotic rapamycin. It would be desirable to find fully synthetic compounds, which inhibit mTOR that are more potent and exhibit improved pharmacokinetic behaviors.

As explained above, PI3K inhibitors and mTOR inhibitors are expected to be novel types of medicaments useful against cell proliferation disorders, especially as carcinostatic agents. Thus, it would be advantageous to have new PI3K inhibitors and mTOR inhibitors as potential treatment regimens for mTOR- and PI3K-related diseases. The instant invention is directed to these and other important ends.

### SUMMARY OF THE INVENTION

In one aspect, the invention provides a compound of the Formula I:

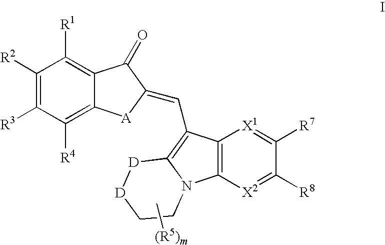

or a pharmaceutically acceptable salt thereof, wherein the constituent variables are as defined below.

In other aspects, the invention provides pharmaceutical compositions comprising compounds or pharmaceutically acceptable salts of compounds of the present formula I.

In one aspect, the compounds or pharmaceutically acceptable salts thereof of the present formula I are useful as mTOR inhibitors.

In one aspect, the compounds or pharmaceutically acceptable salts thereof of the present formula I are useful as PI3K inhibitors.

In one aspect, the compounds or pharmaceutically acceptable salts thereof of the present formula I are useful as mTOR inhibitors and as PI3K inhibitors simultaneously.

In one aspect, the invention provides methods for treating an mTOR-related disorder, comprising administering to a mammal in need thereof, the compounds or pharmaceutically acceptable salts of compounds of the present formula I in an amount effective to treat an mTOR-related disorder.

In one aspect, the invention provides methods for treating a PI3K-related disorder, comprising administering to a mammal in need thereof the compounds or pharmaceutically acceptable salts of compounds of the present formula I in an amount effective to treat a PI3K-related disorder.

In one aspect, the invention provides a compound of the Formula II:

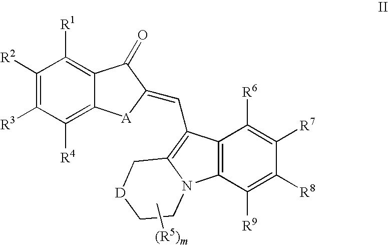

or a pharmaceutically acceptable salt thereof, wherein the constituent variables are as defined below.

In other aspects, the invention provides pharmaceutical compositions comprising compounds or pharmaceutically acceptable salts of compounds of the present formula II.

In one aspect, the compounds or pharmaceutically acceptable salts thereof of the present formula II are useful as mTOR inhibitors.

In one aspect, the compounds or pharmaceutically acceptable salts thereof of the present formula II are useful as PI3K inhibitors.

In one aspect, the compounds or pharmaceutically acceptable salts thereof of the present formula II are useful as mTOR inhibitors and as PI3K inhibitors simultaneously.

In one aspect, the invention provides methods for treating an mTOR-related disorder, comprising administering to a mammal in need thereof, the compounds or pharmaceutically acceptable salts of compounds of the present formula II in an amount effective to treat an mTOR-related disorder.

In one aspect, the invention provides methods for treating a PI3K-related disorder, comprising administering to a mammal in need thereof the compounds or pharmaceutically acceptable salts of compounds of the present formula II in an amount effective to treat a PI3K-related disorder.

In other aspects, the invention provides further methods of synthesizing the compounds or pharmaceutically acceptable salts of compounds of the present formula II.

### DETAILED DESCRIPTION

In one aspect, the invention provides a compound of the Formula I:

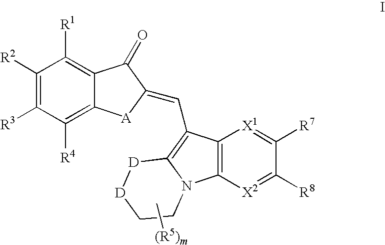

or a pharmaceutically acceptable salt thereof, wherein

### A is —O— or —S—;

### X¹ is N or C—R⁶;

### X² is N or C—R⁹;

with the proviso that at most one of X¹ and X² can be N;
R¹ is H, C₁-C₆alkyl, hydroxyl, C₁-C₆alkyl-NHC(O)NH—, C₂-C₁₀alkenyl-NHC(O)NH—, C₂-C₁₀alkynyl-NHC(O)NH—, C₁-C₆hydroxylalkyl-NHC(O)NH—, amino(C₁-C₆alkyl)-NHC(O)NH—, or C₁-C₆alkoxy;
R² is H, C₁-C₆alkyl, hydroxyl, C₁-C₆alkyl-NHC(O)NH—, C₂-C₁₀alkenyl-NHC(O)NH—, C₂-C₁₀alkynyl-NHC(O)NH—, C₁-C₆hydroxylalkyl-NHC(O)NH—, amino(C₁-C₆alkyl)-NHC(O)NH—, or C₁-C₆alkoxy;
R³ is H, C₁-C₆alkyl, hydroxyl, C₁-C₆alkyl-NHC(O)NH—, C₂-C₁₀alkenyl-NHC(O)NH—, C₂-C₁₀alkynyl-NHC(O)NH—, C₁-C₆hydroxylalkyl-NHC(O)NH—, amino(C₁-C₆alkyl)-NHC(O)NH—, or C₁-C₆alkoxy;
R⁴ is H, C₁-C₆alkyl, hydroxyl, C₁-C₆alkyl-NHC(O)NH—, C₂-C₁₀alkenyl-NHC(O)NH—, C₂-C₁₀alkynyl-NHC(O)NH—, C₁-C₆hydroxylalkyl-NHC(O)NH—, amino(C₁-C₆alkyl)-NHC(O)NH—, or C₁-C₆alkoxy;

R⁶ is H, C₁-C₆alkyl, hydroxyl, or C₁-C₆alkoxy;

R⁷ is H, C₁-C₆alkyl, hydroxyl, or C₁-C₆alkoxy;

R⁸ is H, C₁-C₆alkyl, hydroxyl, or C₁-C₆alkoxy;

R⁹ is H, C₁-C₆alkyl, hydroxyl, or C₁-C₆alkoxy;

one of D is —O—, —N(R¹⁰)—, (CH₂)ₙ, or —S(O)ₒ— and the other D is CH₂;
m is 0, 1, 2, or 3;
n, and o are independently 0, 1, or 2;
R¹⁰ is H, (C₁-C₆alkoxy)carbonyl, C₁-C₆alkyl, (C₁-C₆alkyl)amido, C₁-C₉heterocycle, C₃-C₈cycloalkyl, or C₆-C₁₄aryl;
R⁵ are independently C₁-C₆alkyl, hydroxyl, or C₁-C₆alkoxy;
or two R⁵ groups on the same carbon atom, when taken together with the carbon to which they are attached, can form a carbonyl (C═O) group.

In one aspect, the invention provides a compound of the Formula II:

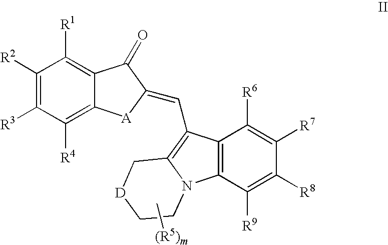

or a pharmaceutically acceptable salt thereof, wherein

### A is —O— or —S—;

R¹ is H, C₁-C₆alkyl, hydroxyl, C₁-C₆alkyl-NHC(O)NH—, C₂-C₁₀alkenyl-NHC(O)NH—, C₂-C₁₀alkynyl-NHC(O)NH—, C₁-C₆hydroxylalkyl-NHC(O)NH—, amino(C₁-C₆alkyl)-NHC(O)NH—, or C₁-C₆alkoxy;
R² is H, C₁-C₆alkyl, hydroxyl, C₁-C₆alkyl-NHC(O)NH—, C₂-C₁₀alkenyl-NHC(O)NH—, C₂-C₁₀alkynyl-NHC(O)NH—, C₁-C₆hydroxylalkyl-NHC(O)NH—, amino(C₁-C₆alkyl)-NHC(O)NH—, or C₁-C₆alkoxy;
R³ is H, C₁-C₆alkyl, hydroxyl, C₁-C₆alkyl-NHC(O)NH—, C₂-C₁₀alkenyl-NHC(O)NH—, C₂-C₁₀alkynyl-NHC(O)NH—, C₁-C₆hydroxylalkyl-NHC(O)NH—, amino(C₁-C₆alkyl)-NHC(O)NH—, or C₁-C₆alkoxy;
R⁴ is H, C₁-C₆alkyl, hydroxyl, C₁-C₆alkyl-NHC(O)NH—, C₂-C₁₀alkenyl-NHC(O)NH—, C₂-C₁₀alkynyl-NHC(O)NH—, C₁-C₆hydroxylalkyl-NHC(O)NH—, amino(C₁-C₆alkyl)-NHC(O)NH—, or C₁-C₆alkoxy;
R⁶ is H, C₁-C₆alkyl, hydroxyl, or C₁-C₆alkoxy;
R⁷ is H, C₁-C₆alkyl, hydroxyl, or C₁-C₆alkoxy;
R⁸ is H, C₁-C₆alkyl, hydroxyl, or C₁-C₆alkoxy;
R⁹ is H, C₁-C₆alkyl, hydroxyl, or C₁-C₆alkoxy;
D is —O—, —N(R¹⁰)—, (CH₂)ₙ, or —S(O)ₒ—;
m is 0, 1, 2, or 3;
n, and o are independently 0, 1, or 2;
R¹⁰ is H, (C₁-C₆alkoxy)carbonyl, C₁-C₆alkyl, (C₁-C₆alkyl)amido, C₁-C₉heterocycle, C₃-C₈cycloalkyl, or C₆-C₁₄aryl;
R⁵ are independently C₁-C₆alkyl, hydroxyl, or C₁-C₆alkoxy;
or two R⁵ groups on the same carbon atom, when taken together with the carbon to which they are attached, can form a carbonyl (C═O) group.

In another aspect of the compound of Formula II, D is oxygen, as shown in Formula III:

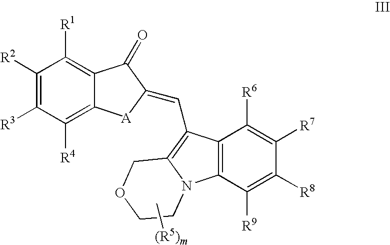

In another aspect of the compound of Formula II, D is N(R¹⁰), as shown in Formula IV:

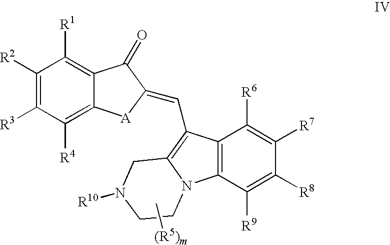

In another aspect of the compound of Formula II, D is (CH₂)ₙ, as shown in Formula V:

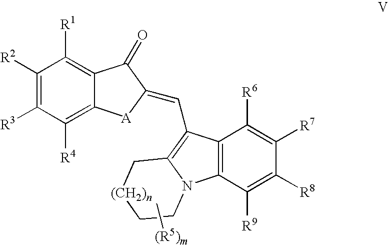

Representative “pharmaceutically acceptable salts” include but are not limited to, e.g., water-soluble and water-insoluble salts, such as the acetate, aluminum, amsonate (4,4-diaminostilbene-2,2-disulfonate), benzathine (N,N′-dibenzylethylenediamine), benzenesulfonate, benzoate, bicarbonate, bismuth, bisulfate, bitartrate, borate, bromide, butyrate, calcium, calcium edetate, camsylate (camphorsulfonate), carbonate, chloride, choline, citrate, clavulariate, diethanolamine, dihydrochloride, diphosphate, edetate, edisylate (camphorsulfonate), esylate (ethanesulfonate), ethylenediamine, fumarate, gluceptate (glucoheptonate), gluconate, glucuronate, glutamate, hexafluorophosphate, hexylresorcinate, hydrabamine (N,N′-bis(dehydroabietyl)ethylenediamine), hydrobromide, hydrochloride, hydroxynaphthoate, 1-hydroxy-2-naphthoate, 3-hydroxy-2-naphthoate, iodide, isothionate (2-hydroxyethanesulfonate), lactate, lactobionate, laurate, lauryl sulfate, lithium, magnesium, malate, maleate, mandelate, meglumine (1-deoxy-1-(methylamino)-D-glucitol), mesylate, methyl bromide, methylnitrate, methylsulfate, mucate, napsylate, nitrate, N-methylglucamine ammonium salt, oleate, oxalate, palmitate, pamoate (4,4′-methylenebis-3-hydroxy-2-naphthoate, or embonate), pantothenate, phosphate, picrate, polygalacturonate, potassium, propionate, p-toluenesulfonate, salicylate, sodium, stearate, subacetate, succinate, sulfate, sulfosaliculate, suramate, tannate, tartrate, teoclate (8-chloro-3,7-dihydro-1,3-dimethyl-1H-purine-2,6-dione), triethiodide, tromethamine (2-amino-2-(hydroxymethyl)-1,3-propanediol), valerate, and zinc salts.

The invention also includes pharmaceutical compositions comprising an effective amount of a [a]-fused indole compound of Formulas I-V and a pharmaceutically acceptable carrier. The compound may be provided as a pharmaceutically acceptable prodrug, hydrated salt, such as a pharmaceutically acceptable salt, or mixtures thereof.

In other aspects, the invention provides pharmaceutical compositions comprising compound of Formulas I-V; a second compound selected from the group consisting of a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, and lavendustin A; and a pharmaceutically acceptable carrier.

In other aspects, the second compound is Avastin.

An “effective amount” when used in connection with a [a]-fused indole compound of this invention is an amount effective for inhibiting mTOR or PI3K in a subject.

In one aspect the invention includes compounds of Formula II in which:

A is —O—;

R¹ is H or hydroxyl;

R² is H;

R² is C₁-C₆alkyl-NHC(O)NH—;

R³ is hydroxyl;

R⁴ is H;

R⁶ is H;

R⁷ is C₁-C₆alkoxy;

R⁷ is methoxy;

R⁸ is H; and/or

R⁹ is H.

In one aspect, A is —O—, R¹ is H or hydroxyl, R² is H, R³ is hydroxyl, R⁴ is H, R⁶ is H, R⁷ is methoxy, R⁸ is H, and R⁹ is H.

In one aspect of the compounds of Formula III, m is 0. In another aspect, m is 2.

In one aspect of the compounds of Formula III, m is 2, and two R⁵ groups are on the same carbon atom and, together with the carbon to which they are attached, form a carbonyl (C═O) group. In a further aspect, this carbonyl (C═O) group is on the 1-position of the 3,4-dihydro-1H-[1,4]oxazino[4,3-a]indole ring.

Illustrative compounds of Formula III include the following compounds:
10-[(Z)-(4,6-dihydroxy-3-oxo-1-benzofuran-2(3H)-ylidene)methyl]-8-methoxy-3,4-dihydro-1H-[1,4]oxazino[4,3-a]indol-1-one;
10-[(Z)-(6-hydroxy-3-oxo-1-benzofuran-2(3H)-ylidene)methyl]-8-methoxy-3,4-dihydro-1H-[1,4]oxazino[4,3-a]indol-1-one; and
(2Z)-4,6-dihydroxy-2-[(8-methoxy-3,4-dihydro-1H-[1,4]oxazino[4,3-a]indol-10-yl)methylene]-1-benzofuran-3(2H)-one.

In one aspect the invention includes compounds of the Formula IV in which:

A is —O—;

R¹ is H or hydroxyl;

R² is H;

R³ is hydroxyl;

R⁴ is H;

R⁶ is H;

R⁷ is C₁-C₆alkoxy;

R⁷ is methoxy;

R⁸ is H; and/or

R⁹ is H.

In one aspect, A is —O—, R¹ is H or hydroxyl, R² is H, R³ is hydroxyl, R⁴ is H, R⁶ is H, R⁷ is methoxy, R⁸ is H, and R⁹ is H.

In one aspect, R¹⁰ is H or C₁-C₆alkyl.

In one aspect, R¹⁰ is H.

In one aspect, R¹⁰ is methyl.

In one aspect, R³ is hydroxyl, R⁷ is methoxy, R¹⁰ is H, and m is 0.

In one aspect, A is —O—, R¹ is H, R² is H, R³ is hydroxyl, R⁴ is H, R⁶ is H, R⁷ is methoxy, R⁸ is H, R⁹ is H, R¹⁰ is H, and m is 0.

Illustrative compounds of Formula IV include the following compounds:
(2Z)-4,6-dihydroxy-2-[(8-methoxy-1,2,3,4-tetrahydropyrazino[1,2-a]indol-10-yl)methylene]-1-benzofuran-3(2H)-one;
(2Z)-6-hydroxy-2-[(8-methoxy-1,2,3,4-tetrahydropyrazino[1,2-a]indol-10-yl)methylene]-1-benzofuran-3(2H)-one;
10-[(Z)-(4,6-dihydroxy-3-oxo-1-benzofuran-2(3H)-ylidene)methyl]-8-methoxy-3,4-dihydropyrazino[1,2-a]indol-1(2H)-one;
(2Z)-4,6-dihydroxy-2-[(8-methoxy-2-methyl-1,2,3,4-tetrahydropyrazino[1,2-a]indol-10-yl)methylene]-1-benzofuran-3(2H)-one; and
10-[(Z)-(4,6-dihydroxy-3-oxo-1-benzofuran-2(3H)-ylidene)methyl]-8-methoxy-2-methyl-3,4-dihydropyrazino[1,2-a]indol-1(2H)-one.

In one aspect the invention includes compounds of the Formula V in which:

A is —O—;

R¹ is H or hydroxyl;

R² is H;

R³ is hydroxyl;

R⁴ is H;

R⁶ is H;

R⁷ is C₁-C₆alkoxy;

R⁷ is methoxy;

R⁸ is H; and/or

R⁹ is H.

In one aspect, A is —O—, R¹ is H or hydroxyl, R² is H, R³ is hydroxyl, R⁴ is H, R⁶ is H, R⁷ is methoxy, R⁸ is H, and R⁹ is H.

In one aspect, n is 0.

In one aspect, m is 0.

An illustrative compound of Formula V is (2Z)-6-hydroxy-2-[(7-methoxy-2,3-dihydro-1H-pyrrolo[1,2-a]indol-9-yl)methylene]-1-benzofuran-3(2H)-one.

In another aspect, the invention provides methods of synthesizing compounds of the Formula II comprising reacting the keto heterocycle 1 with an [a]-fused indole aldehyde of the formula 2:

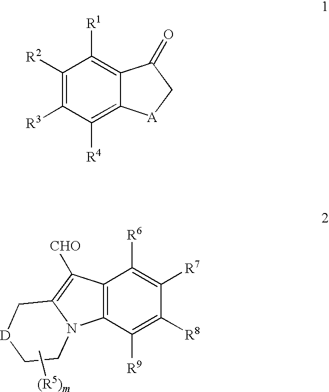

wherein R¹-R⁹, A, D, and m are as defined in Formula II, under acidic conditions effective to condense the aldehyde functional group at position 10 of the [a]-fused indole with the aromatic ketone moiety 1, to give the [a]-fused indole II:

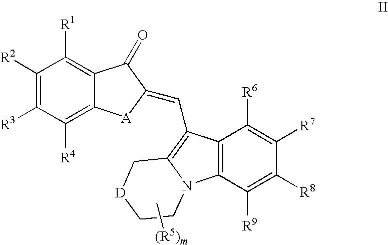

or a pharmaceutically acceptable salt thereof.

In another aspect, the invention provides methods of synthesizing compounds of the Formula II further comprising reacting the tricyclic intermediate of Formula 41 with POCl₃ and DMF formylating the free position on the indole ring

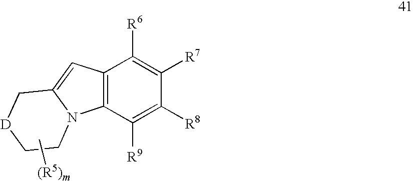

thereby producing 2:

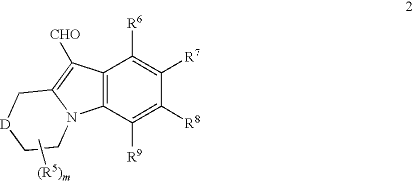

under conditions sufficient to replace the hydrogen atom on the indole ring with a formal radical.

In another aspect, the invention provides methods of synthesizing compounds of the Formula II when D is O or S(O)ₒ wherein o is as defined in Formula II further comprising: (a) reacting the indole ester of Formula 39 with the alkylating agent shown, where X is halogen;

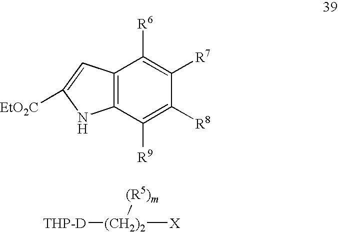

under conditions effective to replace the hydrogen atom on the nitrogen atom at position 1 of the indole ring; followed by removal of the protecting group and ring closure thereby producing 40;

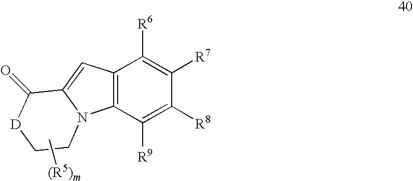

(b) reacting lactone or thiolactone 40 with DIBAL producing an intermediate 3,4-dihydro-1H-[1,4]oxazino[4,3-a]indol-1-ol or 3,4-dihydro-1H-[1,4]thiazino[4,3-a]indol-1-ol; c) reacting the hemiacetal produced with a trialkylsilyl hydride

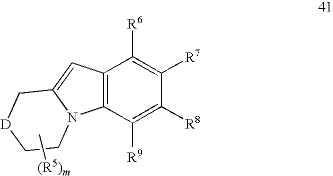

producing the intermediate 41 lacking a carbonyl group.

In another aspect, the invention provides methods of synthesizing compounds of the Formula II when D is N(R¹⁰) wherein R¹⁰ is as defined in Formula II further comprising: (a) reacting the indole ester of Formula 39 with the alkylating agent shown where X is halogen;

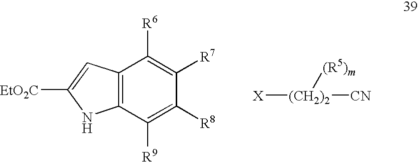

under conditions effective to replace the hydrogen atom on the nitrogen atom at position 1 of the indole ring; followed by reduction and cyclization of the nitrile intermediate producing 42;

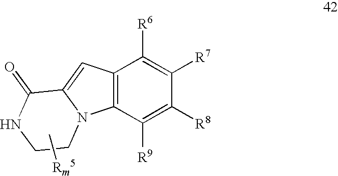

(b) optionally reacting lactam 42 with alkylating agent R¹⁰—X, where X is halogen, producing an intermediate lactam 43;

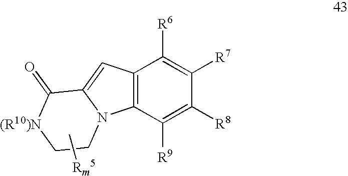

c) reducing the lactam with LAH producing the intermediate 41:

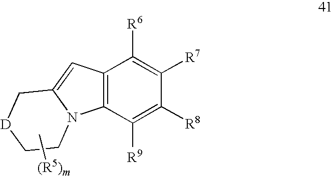

under conditions sufficient to remove the oxygen atom from the carbonyl group.

In another aspect, the invention provides methods of synthesizing compounds of the Formula II when D is (CH₂)ₙ further comprising: (a) reacting the indole ester of Formula 39 with the alkylating agent shown where X is halogen;

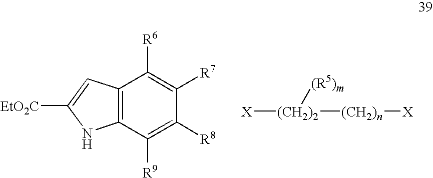

under conditions effective to replace the hydrogen atom on the nitrogen atom at position 1 of the indole ring thereby producing 44;

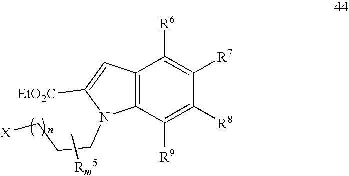

(b) reducing ester 44 with DIBAL, producing an intermediate allylic alcohol; c) oxidizing the alcohol with MnO₂ to make an aldehyde; d) condensing the aldehyde with propane-1,3-dithiol producing the 1,3-dithiane 45;

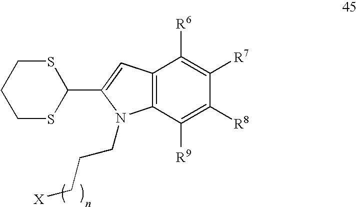

affecting ring closure under basic conditions producing the intermediate 46;

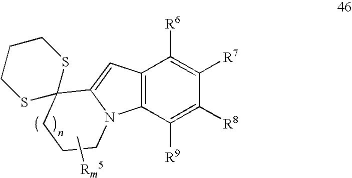

e) removing the dithiane masking group thereby

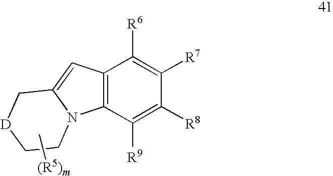

making tricyclic intermediate 41 lacking a carbonyl group.

### DEFINITIONS

The following definitions are used in connection with the [a]-fused indole compounds of the present invention, unless the context indicates otherwise. In general, the number of carbon atoms present in a given group is designated “Cₓ-C_{y}”, where x and y are the lower and upper limits, respectively. For example, a group designated as “C₁-C₆” contains from 1 to 6 carbon atoms. The carbon number as used in the definitions herein refers to carbon backbone and carbon branching, but does not include carbon atoms of the substituents, such as alkoxy substitutions and the like.

“Acyl” refers to a carbonyl group bonded to a moiety comprising from 1 to 8 carbon atoms in a straight, branched, or cyclic configuration or a combination thereof, attached to the parent structure through the carbonyl functionality. The moiety may be saturated or unsaturated, aliphatic or aromatic, and carbocyclic or heterocyclic. One or more carbons in the moiety may be replaced by oxygen, nitrogen (e.g., carboxyamido), or sulfur as long as the point of attachment to the parent remains at the carbonyl. Examples of C₁-C₈acyl include acetyl-, benzoyl-, nicotinoyl, propionyl-, isobutyryl-, oxalyl-, t-butoxycarbonyl-, benzyloxycarbonyl, morpholinylcarbonyl, and the like. An acyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl.

“Alkenyl” refers to a straight or branched chain unsaturated hydrocarbon containing 2-10 carbon atoms and at least one double bond. Examples of a C₂-C₁₀alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, isohexene, 1-heptene, 2-heptene, 3-heptene, 1-octene, 2-octene, 3-octene, 4-octene, 1-nonene, 2-nonene, 3-nonene, 4-nonene, 1-decene, 2-decene, 3-decene, 4-decene and 5-decene. An alkenyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl.

“Alkoxy” refers to the group R—O— where R is an alkyl group, as defined below. Exemplary C₁-C₆alkoxy groups include but are not limited to methoxy, ethoxy, n-propoxy, 1-propoxy, n-butoxy and t-butoxy. An alkoxy group can be unsubstituted or substituted with one or more of the following groups: halogen, hydroxyl, C₁-C₆alkoxy, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —O(C₁-C₆alkyl), —C(O)OH, —C(O)O(C₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆carboxyamidoalkyl-, or —NO₂.

“(Alkoxy)carbonyl” refers to the group alkyl-O—C(O)—. An (alkoxy)carbonyl group can be unsubstituted or substituted with one or more of the following groups: halogen, hydroxyl, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —O(C₁-C₆alkyl), —C(O)OH, —C(O)O(C₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆carboxyamidoalkyl-, or —NO₂. Exemplary (C₁-C₆alkoxy)carbonyl groups include but are not limited to CH₃—O—C(O)—, CH₃CH₂—O—C(O)—, CH₃CH₂CH₂—O—C(O)—, (CH₃)₂CH—O—C(O)—, and CH₃CH₂CH₂CH₂—O—C(O)—.

“Alkyl” refers to a hydrocarbon group that may be a straight chain or branched chain, containing the indicated number of carbon atoms. For example, C₁-C₁₀ indicates that the group has from 1 to 10 (inclusive) carbon atoms in it. In the absence of any numerical designation, “alkyl” indicates 1 to 6 (inclusive) carbon atoms. Examples of C₁-C₆ alkyl groups include, but are not limited to, methyl, ethyl, propyl, butyl, pentyl, hexyl, isopropyl, isobutyl, sec-butyl, tert-butyl, isopentyl, neopentyl, and isohexyl. An alkyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆carboxyamidoalkyl-, or —NO₂.

“(Alkyl)amido-” refers to a —C(O)NH— group in which the nitrogen atom of said group is attached to an alkyl group, as defined above. Representative examples of a (C₁-C₆alkyl)amido group include, but are not limited to, —C(O)NHCH₃, —C(O)NHCH₂CH₃, —C(O)NHCH₂CH₂CH₃, —C(O)NHCH₂CH₂CH₂CH₃, —C(O)NHCH₂CH₂CH₂CH₂CH₃, —C(O)NHCH(CH₃)₂, —C(O)NHCH₂CH(CH₃)₂, —C(O)NHCH(CH₃)CH₂CH₃, —C(O)NH—C(CH₃)₃ and —C(O)NHCH₂C(CH₃)₃.

“(Alkyl)amino-” refers to an —NH-alkyl group, where alkyl is as defined above. Representative examples of an (C₁-C₆alkyl)amino group include, but are not limited to —NHCH₃, —NHCH₂CH₃, —NHCH₂CH₂CH₃, —NHCH₂CH₂CH₂CH₃, —NHCH(CH₃)₂, —NHCH₂CH(CH₃)₂, —NHCH(CH₃)CH₂CH₃ and —NH—C(CH₃)₃. An (alkyl)amino group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆-carboxyamidoalkyl-, or —NO₂.

“Alkylcarboxy” refers to an alkyl group as defined above, attached to the parent structure through the oxygen atom of a carboxyl (C(O)—O—) functionality. Examples of C₁-C₆alkylcarboxy include, but are not limited to, acetoxy, ethylcarboxy, propylcarboxy, and isopentylcarboxy.

“(Alkyl)carboxyamido-” refers to an —NHC(O)— group in which the carbonyl carbon atom of said group is attached to an alkyl group, as defined above. Representative examples of a (C₁-C₆alkyl)carboxyamido group include, but are not limited to, —NHC(O)CH₃, —NHC(O)CH₂CH₃, —NHC(O)CH₂CH₂CH₃, —NHC(O)CH₂CH₂CH₂CH₃, —NHC(O)CH₂CH₂CH₂CH₂CH₃, —NHC(O)CH(CH₃)₂, —NHC(O)CH₂CH(CH₃)₂, —NHC(O)CH(CH₃)CH₂CH₃, —NHC(O)—C(CH₃)₃ and —NHC(O)CH₂C(CH₃)₃.

“Alkylene”, “alkenylene”, and “alkynylene” refers to the subsets of alkyl, alkenyl and alkynyl groups, as defined herein, including the same residues as alkyl, alkenyl, and alkynyl, but having two points of attachment within a chemical structure. Examples of C₁-C₆alkylene include methylene (—CH₂—), ethylene (—CH₂CH₂—), propylene (—CH₂CH₂CH₂—), and dimethylpropylene (—CH₂C(CH₃)₂CH₂—). Likewise, examples of C₂-C₆alkenylene include ethenylene (—CH═CH— and propenylene (—CH═CH—CH₂—). Examples of C₂-C₆alkynylene include ethynylene (—C≡C—) and propynylene (—C≡C—CH₂—).

“Alkylthio” refers to groups of straight chain or branched chain with 1 to 6 carbon atoms, attached to the parent structure through a sulfur atom. Examples of a C₁-C₆alkylthio group include methylthio, ethylthio, n-propylthio, i-propylthio, n-butylthio, i-butylthio, s-butylthio, t-butylthio, n-pentylthio, and n-hexylthio.

“Alkynyl” refers to a straight or branched chain unsaturated hydrocarbon containing 2-10 carbon atoms, respectively, and at least one triple bond. Examples of a C₂-C₁₀alkynyl group include, but are not limited to, acetylene, propyne, 1-butyne, 2-butyne, isobutyne, sec-butyne, 1-pentyne, 2-pentyne, isopentyne, 1-hexyne, 2-hexyne, 3-hexyne, isohexyne, 1-heptyne, 2-heptyne, 3-heptyne, 1-octyne, 2-octyne, 3-octyne, 4-octyne, 1-nonyne, 2-nonyne, 3-nonyne, 4-nonyne, 1-decyne, 2-decyne, 3-decyne, 4-decyne and 5-decyne. A alkynyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl.

“Amido(aryl)-” refers to an aryl group, as defined below, wherein one of the aryl group's hydrogen atoms has been replaced with one or more —C(O)NH₂ groups. Representative examples of an amido(C₆-C₁₄aryl)- group include 2-C(O)NH₂-phenyl, 3-C(O)NH₂-phenyl, 4-C(O)NH₂-phenyl, 1-C(O)NH₂-naphthyl, and 2-C(O)NH₂-naphthyl.

“Amino(alkyl)-” refers to an alkyl group, as defined above, wherein one or more of the alkyl group's hydrogen atoms has been replaced with —NH₂. Representative examples of an amino(C₁-C₆alkyl) group include, but are not limited to —CH₂NH₂, —CH₂CH₂NH₂, —CH₂CH₂CH₂ NH₂, —CH₂CH₂CH₂CH₂NH₂, —CH₂CH(NH₂)CH₃, —CH₂CH(NH₂)CH₂CH₃, —CH(NH₂)CH₂CH₃ and —C(CH₃)₂(CH₂NH₂), —CH₂CH₂CH₂CH₂CH₂NH₂, and —CH₂CH₂CH(NH₂)CH₂CH₃. An amino(alkyl) group can be unsubstituted or substituted with one or two of the following groups C₁-C₆alkoxy, C₆-C₁₄aryl, C₁-C₉heteroaryl, C₃-C₈cycloalkyl, and C₁-C₆alkyl.

“Aryl” refers to an aromatic hydrocarbon group. If not otherwise specified, in this specification the term aryl refers to a C₆-C₁₄aryl group. Examples of an C₆-C₁₄aryl group include, but are not limited to, phenyl, 1-naphthyl, 2-naphthyl, 3-biphen-1-yl, anthryl, tetrahydronaphthyl, fluorenyl, indanyl, biphenylenyl, and acenaphthenyl, groups. An aryl group can be unsubstituted or substituted with one or more of the following groups: C₁-C₆alkyl, C₃-C₈cycloalkyl, C₁-C₆ perfluoroalkyl-, halo, haloalkyl-, hydroxyl, C₁-C₆hydroxylalkyl-, —NH₂, aminoalkyl-, dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“(Aryl)alkyl” refers to an alkyl group, as defined above, wherein one or more of the alkyl group's hydrogen atoms has been replaced with a C₆-C₁₄aryl group as defined above. (C₆-C₁₄Aryl)alkyl moieties include benzyl, 1-phenylethyl, 2-phenylethyl, 3-phenylpropyl, 2-phenylpropyl, 1-naphthylmethyl, 2-naphthylmethyl and the like. An (aryl)alkyl group can be unsubstituted or substituted with one or more of the following groups: halogen, —NH₂, hydroxyl, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆-carboxyamidoalkyl-, or —NO₂.

“(Aryl)amino” refers to a radical of formula aryl-NH—, wherein “aryl” is as defined above. Examples of (C₆-C₁₄aryl)amino radicals include, but are not limited to, phenylamino (anilido), 1-naphthlamino, 2-naphthlamino and the like. An (aryl)amino group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl.

“(Aryl)oxy” refers to the group Ar—O— where Ar is an aryl group, as defined above. Exemplary (C₆-C₁₄aryl)oxy groups include but are not limited to phenyloxy, α-naphthyloxy, and β-naphthyloxy. A (aryl)oxy group can be unsubstituted or substituted with one or more of the following groups: C₁-C₆alkyl, halo, haloalkyl-, hydroxyl, C₁-C₆hydroxylalkyl-, —NH₂, aminoalkyl-, -dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“Cycloalkyl” refers to a monocyclic, saturated hydrocarbon ring containing 3-8 carbon atoms. Representative examples of a C₃-C₈cycloalkyl include, but are not limited to, cyclopropyl, cyclobutyl, cyclopentyl, cyclohexyl, cycloheptyl and cyclooctyl. A cycloalkyl can be unsubstituted or independently substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl, haloalkyl-, aminoalkyl-, —OC(O)(C₁-C₆alkyl), C₁-C₆-carboxyamidoalkyl-, or —NO₂. Additionally, each of any two hydrogen atoms on the same carbon atom of the cycloalkyl ring can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle containing two oxygen atoms.

“Bicyclic cycloalkyl” refers to a bicyclic, saturated hydrocarbon ring system containing 6-10 carbon atoms. Representative examples of a C₆-C₁₀bicyclic cycloalkyl include, but are not limited to, cis-1-decalinyl, trans 2-decalinyl, cis-4-perhydroindanyl, and trans-7-perhydroindanyl. A bicyclic cycloalkyl can be unsubstituted or independently substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl, haloalkyl-, aminoalkyl-, —OC(O)(C₁-C₆alkyl), C₁-C₆-carboxyamidoalkyl-, or —NO₂. Additionally, each of any two hydrogen atoms on the same carbon atom of the bicyclic cycloalkyl rings can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle containing two oxygen atoms.

“Carboxyamidoalkyl-” refers to a primary carboxyamide (—CONH₂), a secondary carboxyamide (CONHR′) or a tertiary carboxyamide (CONR′R″), where R′ and R″ are the same or different substituent groups selected from C₁-C₆alkyl, C₂-C₆alkenyl, C₂-C₆alkynyl, C₆-C₁₄aryl, C₁-C₉heteroaryl, or C₃-C₈cycloalkyl, attached to the parent compound by an alkyl group as defined above. Exemplary C₁-C₆-carboxyamidoalkyl- groups include but are not limited to NH₂C(O)—CH₂—, CH₃NHC(O)—CH₂CH₂—, (CH₃)₂NC(O)—CH₂CH₂CH₂—, CH₂═CHCH₂NHC(O)—CH₂CH₂CH₂CH₂—, HCCCH₂NHC(O)—CH₂CH₂CH₂CH₂CH₂—, C₆H₅NHC(O)—CH₂CH₂CH₂CH₂CH₂CH₂—, 3-pyridylNHC(O)—CH₂CH(CH₃)CH₂CH₂—, and cyclopropyl-CH₂NHC(O)—CH₂CH₂C(CH₃)₂CH₂—.

“Cycloalkenyl” refers to non-aromatic, carbocyclic rings containing 3-10 carbon atoms with one or more carbon-to-carbon double bonds within the ring system. The “cycloalkenyl” may be a single ring or may be multi-ring. Multi-ring structures may be bridged or fused ring structures. A cycloalkenyl can be unsubstituted or independently substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl, haloalkyl-, aminoalkyl-, —OC(O)(C₁-C₆alkyl), C₁-C₆-carboxyamidoalkyl-, or —NO₂ Additionally, each of any two hydrogen atoms on the same carbon atom of the cycloalkenyl rings may be replaced by an oxygen atom to form an oxo (═O) substituent or the two hydrogen atoms may be replaced by an alkylenedioxy group so that the alkylenedioxy group, when taken together with the carbon atom to which it is attached, form a 5- to 7-membered heterocycle containing two oxygen atoms. Examples of C₃-C₁₀cycloalkenyls include, but are not limited to, cyclopropenyl, cyclobutenyl, cyclopentenyl, cyclohexenyl, 4,4a-octalin-3-yl, and cyclooctenyl.

“Di(alkyl)amino” refers to a nitrogen atom which has attached to it two alkyl groups, as defined above. Each alkyl group can be independently selected from the alkyl groups. Representative examples of an di(C₁-C₆alkyl)amino- group include, but are not limited to, —N(CH₃)₂, —N(CH₂CH₃)(CH₃), —N(CH₂CH₃)₂, —N(CH₂CH₂CH₃)₂, —N(CH₂CH₂CH₂CH₃)₂, —N(CH(CH₃)₂)₂, —N(CH(CH₃)₂)(CH₃), —N(CH₂CH(CH₃)₂)₂, —NH(CH(CH₃)CH₂CH₃)₂, —N(C(CH₃)₃)₂, —N(C(CH₃)₃)(CH₃), and —N(CH₃)(CH₂CH₃). The two alkyl groups on the nitrogen atom, when taken together with the nitrogen to which they are attached, can form a 3- to 7-membered nitrogen containing heterocycle wherein up to two of the carbon atoms of the heterocycle can be replaced with —N(R)—, —O—, or —S(O)ₒ—. R is hydrogen, C₁-C₆alkyl, C₃-C₈cycloalkyl, C₆-C₁₄aryl, C₁-C₉heteroaryl, amino(C₁-C₆alkyl), or arylamino. Variable o is 0, 1, or 2.

“Halo” of “halogen” refers to —F, —Cl, —Br or —I.

“Haloalkyl” refers to an alkyl group, as defined above, wherein one or more of the alkyl group's hydrogen atoms has been replaced with —F, —Cl, —Br, or —I. Each substitution can be independently selected from —F, —Cl, —Br, or —I. Representative examples of an C₁-C₆haloalkyl group include, but are not limited to —CH₂F, —CCl₃, —CF₃, CH₂CF₃, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂ CH₂CH₂I, —CH₂CH₂CH₂CH₂CH₂Br, —CH₂CH₂CH₂CH₂CH₂I, —CH₂CH(Br)CH₃, —CH₂ CH(Cl)CH₂CH₃, —CH(F)CH₂CH₃ and —C(CH₃)₂(CH₂Cl).

“Heteroaryl” refers to 5-10-membered mono and bicyclic aromatic groups containing at least one heteroatom selected from oxygen, sulfur and nitrogen. Examples of monocyclic C₁-C₅heteroaryl radicals include, but are not limited to, oxazinyl, thiazinyl, diazinyl, triazinyl, tetrazinyl, imidazolyl, tetrazolyl, isoxazolyl, furanyl, furazanyl, oxazolyl, thiazolyl, thiophenyl, pyrazolyl, triazolyl, pyrimidinyl, N-pyridyl, 2-pyridyl, 3-pyridyl and 4-pyridyl. Examples of C₁-C₉bicyclic heteroaryl radicals include but are not limited to, benzimidazolyl, indolyl, isoquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl. A heteroaryl group can be unsubstituted or substituted with one or more of the following groups: C₁-C₆alkyl, halo, haloalkyl-, hydroxyl, C₁-C₆hydroxylalkyl-, —NH₂, aminoalkyl-, dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“(Heteroaryl)oxy” refers to the group Het-O— where Het is a heteroaryl group, as defined above. Exemplary (C₁-C₉heteroaryl)oxy groups include but are not limited to pyridin-2-yloxy, pyridin-3-yloxy, pyrimidin-4-yloxy, and oxazol-5-yloxy. A (heteroaryl)oxy group can be unsubstituted or substituted with one or more of the following groups: C₁-C₆alkyl, halo, haloalkyl-, hydroxyl, C₁-C₆hydroxylalkyl-, —NH₂, aminoalkyl-, dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“Heterocycle” refers to 3-10-membered mono and bicyclic groups containing at least one heteroatom selected from oxygen, sulfur and nitrogen. A heterocycle may be saturated, aromatic, or partially saturated. Exemplary C₁-C₉heterocycle groups include but are not limited to aziridine, oxirane, oxirene, thiirane, pyrroline, pyrrolidine, pyrrole, dihydrofuran, tetrahydrofuran, furan, dihydrothiophene, tetrahydrothiophene, thiophene, pyrazole, imidazole, triazole, tetrazole, oxazole, isoxazole, thiazole, isothiazole, dithiolane, piperidine, pyridine, tetrahydropyran, pyran, thiane, thiine, piperazine, oxazine, thiazine, dithiane, dioxane, pyrazine, pyrimidine, pyridazine, quinoline, isoquinoline, purine, and quinazoline.

The term “heteroatom” refers to a sulfur, nitrogen, or oxygen atom.

“Heterocyclyl(alkyl)” refers to an alkyl group, as defined above, wherein one or more of the alkyl group's hydrogen atoms has been replaced with a heterocycle group as defined above. Heterocyclyl(C₁-C₆alkyl) moieties include 2-pyridylmethyl, 1-piperazinylethyl, 2-thiophenylethyl, 4-morpholinylpropyl, 3-pyridylpropyl, 2-quinolinylmethyl, 2-indolylmethyl, 6-piperazinylhexyl, and the like. A heterocyclyl(alkyl) group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), 4- to 7-membered monocyclic heterocycle, C₆-C₁₄aryl, C₁-C₉heteroaryl, or C₃-C₈cycloalkyl.

“Hydroxylalkyl-” refers to an alkyl group, as defined above, wherein one or more of the alkyl group's hydrogen atoms has been replaced with hydroxyl groups. Examples of C₁-C₆hydroxylalkyl- moieties include, for example, —CH₂OH, —CH₂CH₂OH, —CH₂CH₂CH₂OH, —CH₂CH(OH)CH₂OH, —CH₂CH(OH)CH₃, —CH(CH₃)CH₂OH and higher homologs.

“Hydroxylalkenyl-” refers to a straight or branched chain hydrocarbon, containing 3-6 carbon atoms, and at least one double bond, substituted on one or more sp³ carbon atom with a hydroxyl group. Examples of C₃-C₆hydroxylalkenyl-moieties include chemical groups such as —CH═CHCH₂OH, —CH(CH═CH₂)OH, —CH₂CH═CHCH₂OH, —CH(CH₂CH═CH₂)OH, —CH═CHCH₂CH₂OH, —CH(CH═CHCH₃)OH, —CH═CHCH(CH₃)OH, —CH₂CH(CH═CH₂)OH, and higher homologs.

“Monocyclic heterocycle” refers to a monocyclic aromatic, cycloalkyl, or cycloalkenyl in which 1-4 of the ring carbon atoms have been independently replaced with an N, O or S atom. The monocyclic heterocyclic ring can be attached via a nitrogen, sulfur, or carbon atom. Representative examples of a 3- to 7-membered monocyclic heterocycle group include, but are not limited to, piperidinyl, 1,2,5,6-tetrahydropyridinyl, piperazinyl, morpholinyl, pyrrolyl, oxazinyl, thiazinyl, diazinyl, triazinyl, tetrazinyl, imidazolyl, tetrazolyl, pyrrolidinyl, isoxazolyl, furanyl, furazanyl, pyridinyl, oxazolyl, thiazolyl, thiophenyl, pyrazolyl, triazolyl, and pyrimidinyl. A monocyclic heterocycle group can be unsubstituted or substituted with one or more of the following groups: C₁-C₈acyl, C₁-C₆alkyl, C₁-C₆heterocyclylalkyl, (C₆-C₁₄aryl)alkyl, halo, C₁-C₆haloalkyl-, hydroxyl, C₁-C₆hydroxylalkyl-, —NH₂, aminoalkyl-, -dialkylamino-, —COOH, —C(O)O—(C₁-C₆alkyl), —OC(O)(C₁-C₆alkyl), (C₆-C₁₄aryl)alkyl-O—C(O)—, N-alkylamido-, —C(O)NH₂, (C₁-C₆alkyl)amido-, or —NO₂.

“Bicyclic heterocycle” refers to a bicyclic aromatic, bicyclic cycloalkyl, or bicyclic cycloalkenyl in which 1-4 of the ring carbon atoms have been independently replaced with an N, O or S atom. The bicyclic heterocyclic ring can be attached via a nitrogen, sulfur, or carbon atom. Representative examples of a 6- to 10-membered bicyclic heterocycle group include, but are not limited to, benzimidazolyl, indolyl, indolinyl, isoquinolinyl, indazolyl, quinolinyl, tetrahydroquinolinyl, quinazolinyl, purinyl, benzisoxazolyl, benzoxazolyl, benzthiazolyl, benzodiazolyl, benzotriazolyl, isoindolyl and indazolyl. A bicyclic heterocycle group can be unsubstituted or substituted with one or more of the following groups: C₁-C₈acyl, C₁-C₆alkyl, C₁-C₆heterocyclylalkyl, (C₆-C₁₄aryl)alkyl, halo, C₁-C₆haloalkyl-, hydroxyl, C₁-C₆hydroxylalkyl-, —NH₂, aminoalkyl-, -dialkylamino-, —COOH, —C(O)O—(C₁-C₆alkyl), —OC(O)(C₁-C₆alkyl), (C₆-C₁₄aryl)alkyl-O—C(O)—, N-alkylamido-, —C(O)NH₂, (C₁-C₆alkyl)amido-, or —NO₂.

“Perfluoroalkyl-” refers to a straight or branched chain hydrocarbon having two or more fluorine atoms. Examples of a C₁-C₆ perfluoroalkyl- group include CF₃, CH₂CF₃, CF₂CF₃ and CH(CF₃)₂.

The term “optionally substituted” as used herein means that at least one hydrogen atom of the optionally substituted group has been substituted with halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl.

A “subject” is a mammal, e.g., a human, mouse, rat, guinea pig, dog, cat, horse, cow, pig, or non-human primate, such as a monkey, chimpanzee, baboon or gorilla.

The compounds within the present invention possess double bonds connecting the indole to the benzofuran or benzothiophene nucleus. These double bonds can exist as geometric isomers, and the invention includes both E and Z isomers of such double bonds. All such stable isomers are contemplated in the present invention.

The [a]-fused indole compounds of the present invention exhibit a PI3K inhibitory activity and therefore, can be utilized in order to inhibit abnormal cell growth in which PI3K plays a role. Thus, the [a]-fused indole compounds are effective in the treatment of disorders with which abnormal cell growth actions of PI3K are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the [a]-fused indole compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovary cancer, prostate cancer, lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

The [a]-fused indole compounds of the present invention exhibit an mTOR inhibitory activity and therefore, can be utilized in order to inhibit abnormal cell growth in which mTOR plays a role. Thus, the [a]-fused indole compounds are effective in the treatment of disorders with which abnormal cell growth actions of mTOR are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the [a]-fused indole compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovary cancer, prostate cancer, lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

For therapeutic use, the pharmacologically active compounds of Formulas I-V will normally be administered as a pharmaceutical composition comprising as the (or an) essential active ingredient at least one such compound in association with a solid or liquid pharmaceutically acceptable carrier and, optionally, with pharmaceutically acceptable adjutants and excipients employing standard and conventional techniques.

The pharmaceutical compositions of this invention include suitable dosage forms for oral, parenteral (including subcutaneous, intramuscular, intradermal and intravenous) bronchial or nasal administration. Thus, if a solid carrier is used, the preparation may be tableted, placed in a hard gelatin capsule in powder or pellet form, or in the form of a troche or lozenge. The solid carrier may contain conventional excipients such as binding agents, fillers, tableting lubricants, disintegrants, wetting agents and the like. The tablet may, if desired, be film coated by conventional techniques. If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and like may be utilized. Injectable suspensions also may be used, in which case conventional suspending agents may be employed. Conventional preservatives, buffering agents and the like also may be added to the parenteral dosage forms. Particularly useful is the administration of a compound of Formulas I-V directly in parenteral formulations. The pharmaceutical compositions are prepared by conventional techniques appropriate to the desired preparation containing appropriate amounts of the active ingredient, that is, the compound of Formulas I-V according to the invention. See, for example, Remington: The Science and Practice of Pharmacy, 20th Edition. Baltimore, Md.: Lippincott Williams & Wilkins, 2000.

The dosage of the compounds of Formulas I-V to achieve a therapeutic effect will depend not only on such factors as the age, weight and sex of the patient and mode of administration, but also on the degree of potassium channel activating activity desired and the potency of the particular compound being utilized for the particular disorder of disease concerned. It is also contemplated that the treatment and dosage of the particular compound may be administered in unit dosage form and that one skilled in the art would adjust the unit dosage form accordingly to reflect the relative level of activity. The decision as to the particular dosage to be employed (and the number of times to be administered per day is within the discretion of the physician, and may be varied by titration of the dosage to the particular circumstances of this invention to produce the desired therapeutic effect.

A suitable dose of a compound of Formulas I-V or pharmaceutical composition thereof for a mammal, including man, suffering from, or likely to suffer from any condition as described herein is an amount of active ingredient from about 0.01 mg/kg to 10 mg/kg body weight. For parenteral administration, the dose may be in the range of 0.1 mg/kg to 1 mg/kg body weight for intravenous administration. For oral administration, the dose may be in the range about 0.1 mg/kg to 5 mg/kg body weight. The active ingredient will preferably be administered in equal doses from one to four times a day. However, usually a small dosage is administered, and the dosage is gradually increased until the optimal dosage for the host under treatment is determined.

However, it will be understood that the amount of the compound actually administered will be determined by a physician, in the light of the relevant circumstances including the condition to be treated, the choice of compound of be administered, the chosen route of administration, the age, weight, and response of the individual patient, and the severity of the patient's symptoms.

The amount of the compound of the present invention or a pharmaceutically acceptable salt thereof that is effective for inhibiting mTOR or PI3K in a subject. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one compound of the present invention or a pharmaceutically acceptable salt thereof is administered, the effective dosage amounts correspond to the total amount administered.

In certain aspects, the present invention is directed to prodrugs of the [a]-fused indole compounds or pharmaceutically acceptable salts thereof of the present invention. Various forms of prodrugs are known in the art.

In other aspects, the invention provides a method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof a composition comprising a compound of Formulas I-V; a second compound selected from the group consisting of a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, and lavendustin A; and a pharmaceutically acceptable carrier in an amount effective to treat the cancer.

In other aspects, the invention provides a method of treating lung cancer comprising administering to a mammal in need thereof a composition comprising a compound of Formulas I-V; a second compound selected from the group consisting of cisplatin, carboplatin, gemcitabine, paclitaxel, docetaxel, etoposide (vinorelbine), topotecan, and irinotecan; and a pharmaceutically acceptable carrier in an amount effective to treat the cancer.

In other aspects, the invention provides a method of treating breast cancer comprising administering to a mammal in need thereof a composition comprising a compound of Formulas I-V; a second mixture selected from the group consisting of the TAC combination (Taxotere (docetaxel), Adriamycin (doxorubicin), and Cyclophosphamide) or the FAC (or CAF) combination (5-Fluorouracil, Adriamycin (doxorubicin), and Cyclophosphamide); and a pharmaceutically acceptable carrier in an amount effective to treat the cancer.

In other aspects, the invention provides a method of treating colon cancer comprising administering to a mammal in need thereof a composition comprising a compound of Formulas I-V; a second ingredient selected from the group consisting of the FOLFOX-4 combination (FOLinic Acid (leucovorin), 5-Fluorouracil (5-FU) and OXaliplatin (Eloxatin); or the compounds Capecitabine (Xeloda), Irinotecan (Camptosar), Bevacizumab (Avastin), or Bortezomib (Velcade); and a pharmaceutically acceptable carrier in an amount effective to treat the cancer.

In other aspects, the invention provides a method of treating brain cancer (glioma) comprising administering to a mammal in need thereof a composition comprising a compound of Formulas I-V; a second compound selected from the group consisting of temozolomide; and a pharmaceutically acceptable carrier in conjunction with radiotherapy, in an amount effective to treat the cancer.

In other aspects, the invention provides a method of treating prostate cancer comprising administering to a mammal in need thereof in conjunction with radiotherapy a composition comprising a compound of Formulas I-V; a second compound selected from the group consisting of docetaxel, thalidomide, an anti-androgen, and bevacizumab (avastin); and a pharmaceutically acceptable carrier in an amount effective to treat the cancer.

The invention further comprises a method of treating advanced renal cell carcinoma, comprising administering to a mammal in need thereof the compounds or a pharmaceutically acceptable salt thereof of the present formulas II-V in an amount effective to treat advanced renal cell carcinoma.

Another aspect is a method of treating acute lymphoblastic leukemia, comprising administering to a mammal in need thereof the compounds or a pharmaceutically acceptable salt thereof of any of the present formulas II-V in an amount effective to treat acute lymphoblastic leukemia.

Another aspect is a method of treating acute lymphoblastic leukemia, comprising administering to a mammal in need thereof the compounds or a pharmaceutically acceptable salt thereof of any of the present formulas II-V in an amount effective to treat malignant melanoma.

Another aspect is a method of treating acute lymphoblastic leukemia, comprising administering to a mammal in need thereof the compounds or a pharmaceutically acceptable salt thereof of any of the present formulas II-V in an amount effective to treat soft-tissue or bone sarcoma.

Methods useful for making the [a]-fused indole compounds are set forth in Schemes 1-18 and in the Examples, below:

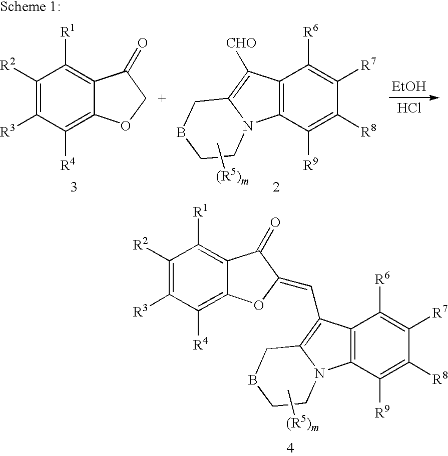

Condensation between ketone 3 and aldehyde 2 was done under acidic conditions, to form compound 4, which is a compound of Formula II in which A is O, Schemes 2-8, below, illustrate methods for making aldehydes of formula 2.

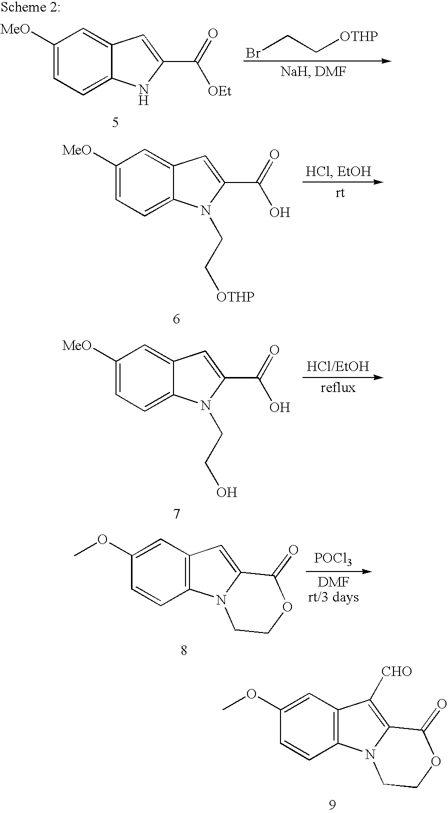

8-Methoxy-1-oxo-3,4-dihydro-1H-[1,4]oxazino[4,3-a]indole-10-carbaldehyde 9 was made from the commercially available ethyl 5-methoxy-1H-indole-2-carboxylate by the four-step sequence shown in Scheme 2.

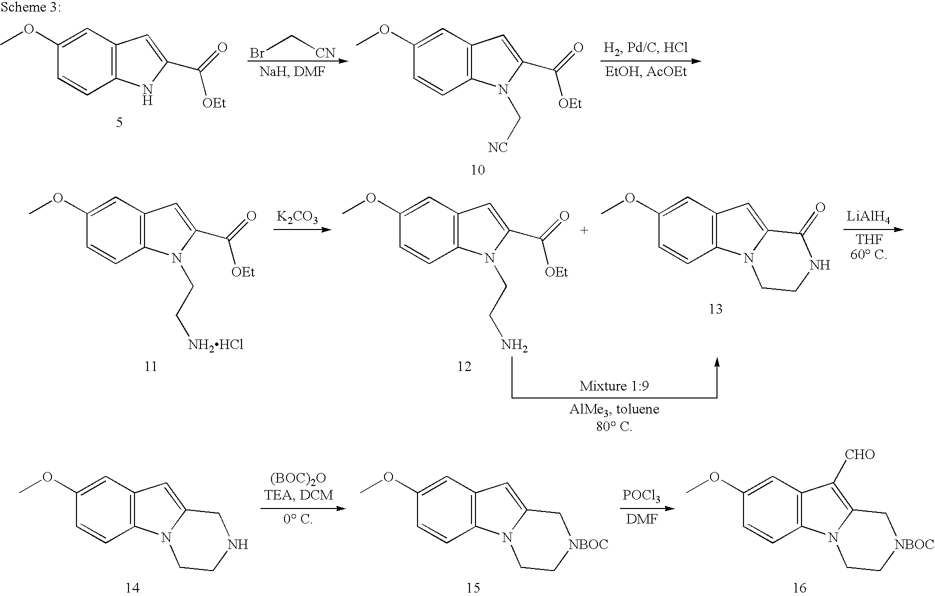

tert-Butyl 10-formyl-8-methoxy-3,4-dihydropyrazino[1,2-a]indole-2(1H)-carboxylate 16 was also made from the commercially available ethyl 5-methoxy-1H-indole-2-carboxylate by the six-step sequence shown in Scheme 3. Uncyclized amino ester 12 was closed to lactam 13 with a Lewis acid and heat.

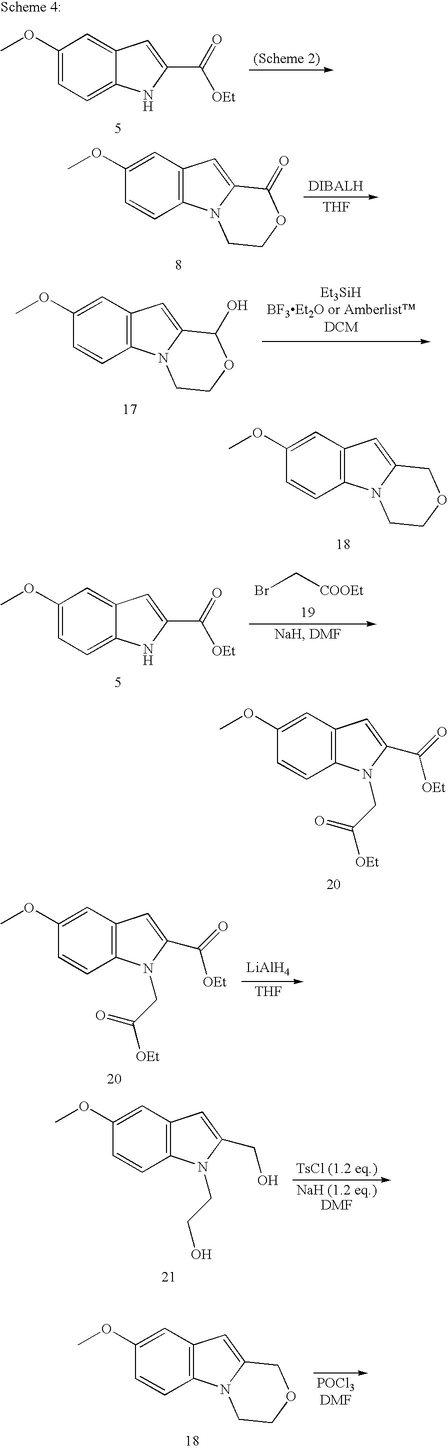

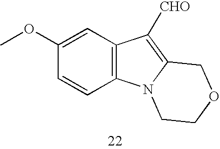

The commercially available ethyl 5-methoxy-1H-indole-2-carboxylate was used to make 8-methoxy-3,4-dihydro-1H-[1,4]oxazino[4,3-a]indole-10-carbaldehyde 22 by the nine-step sequence shown in Scheme 4.

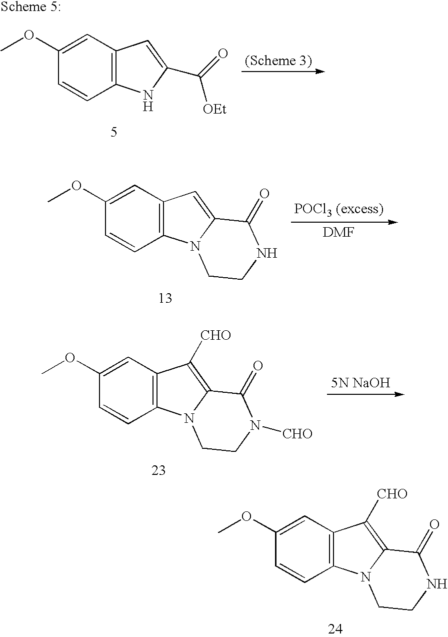

8-Methoxy-1-oxo-1,2,3,4-tetrahydropyrazino[1,2-a]indole-10-carbaldehyde 24 was made by the five-step sequence shown in Scheme 5.

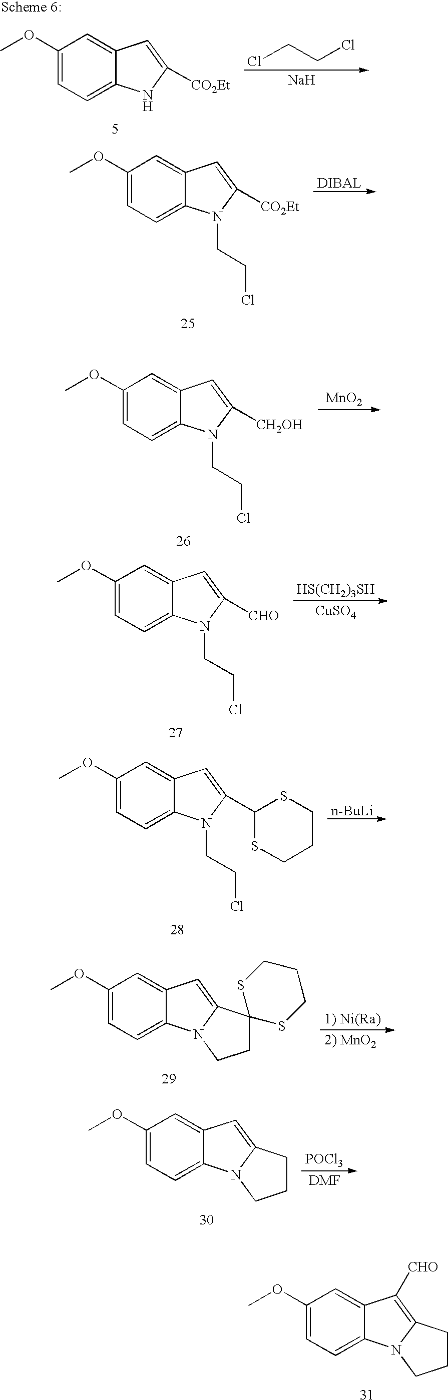

7-Methoxy-2,3-dihydro-1H-pyrrolo[1,2-a]indole-9-carbaldehyde 31 was made by the eight-step sequence shown in Scheme 6.

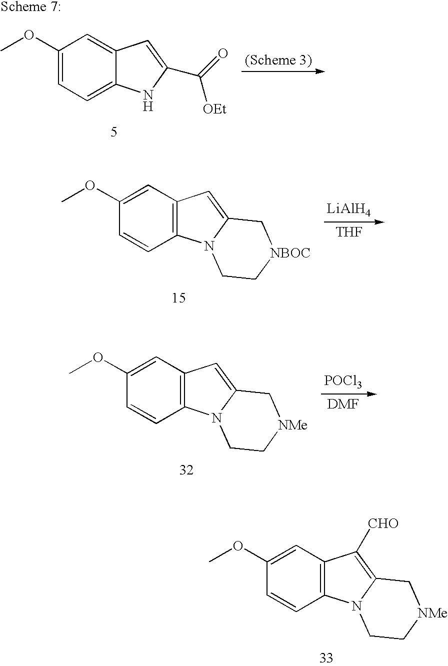

As shown in Scheme 7,8-methoxy-2-methyl-1,2,3,4-tetrahydropyrazino[1,2-a]indole-10-carbaldehyde 33 was made using a variant of the synthesis outlined in Scheme 3. Reduction of the BOC protecting group introduced a methyl at position 2 of the 1,2,3,4-tetrahydropyrazino[1,2-a]indole ring.

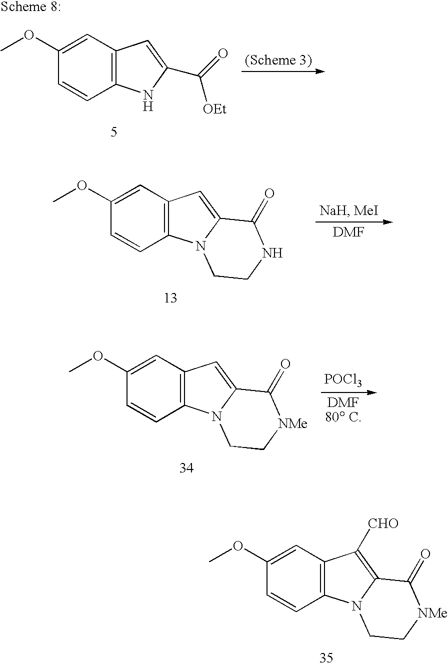

As shown in Scheme 8,8-methoxy-2-methyl-1-oxo-1,2,3,4-tetrahydropyrazino[1,2-a]indole-10-carbaldehyde 35 was made using a variant of the synthesis outlined in Scheme 3. This time alkylation with methyl iodide introduced a methyl at position 2 of the 1,2,3,4-tetrahydropyrazino[1,2-a]indole ring.

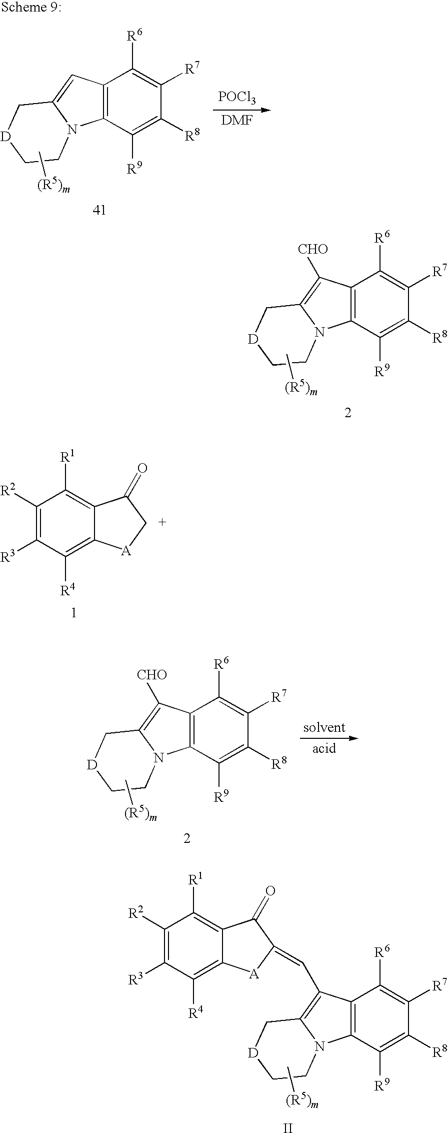

Scheme 9 illustrates the synthesis of compound II by condensation between ketone 1 and aldehyde 2, which was done under acidic conditions. This method was illustrated in Scheme 1 for compounds where A is O, but may also be where A is S. Classical Vilsmeier-Haack procedure with POCl₃ in DMF was used to make aldehyde 2.

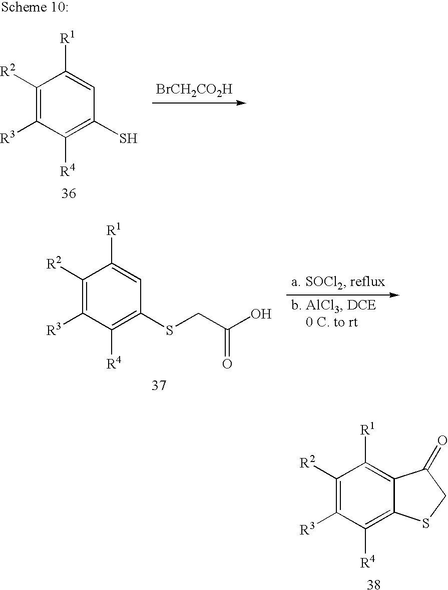

The sulfur-containing ketone 38 required for the synthesis outlined in Scheme 9 where A is S can be made in three steps as shown in Scheme 10.

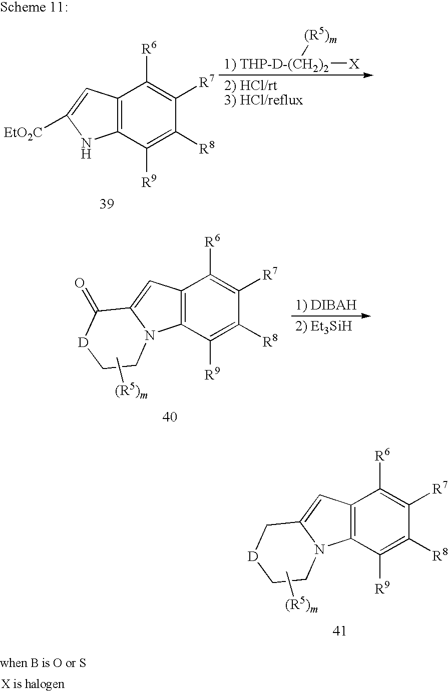

In the case when D is a chalcogen, then the required tricyclic intermediate 41 used in Scheme 9, could be made as shown in Scheme 11.

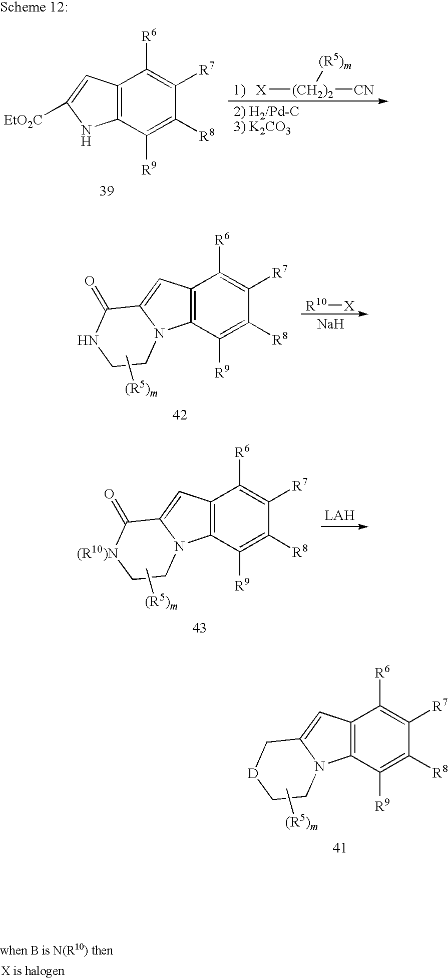

When D contains nitrogen, then the double alkylation procedure shown in Scheme 12 could be used. Alkylation of the lactam 42 would allow for ready introduction of substituent R¹⁰ and ready access to the tricyclic intermediate 41.

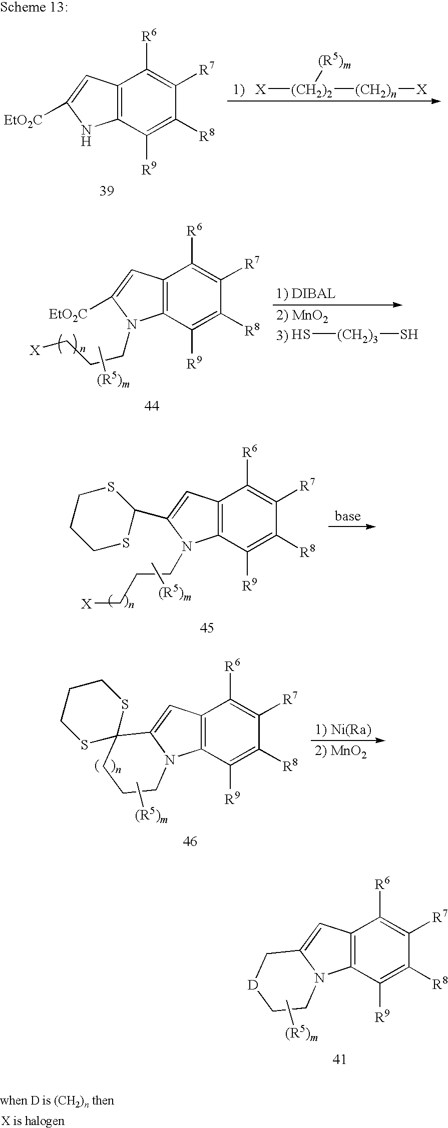

When D contains carbon, then cyclization of the 1,3-dithiane intermediate 45, would, after desulfurization, give the required tricyclic intermediate 41.

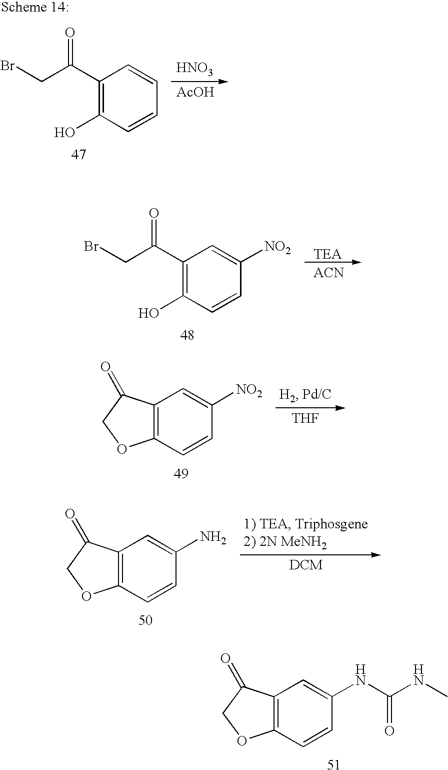

The keto heterocycle 3 with A=O, R¹=R³=R⁴=H, and R²=(CH₃)—NHC(O)NH—, Compound 51, was made as shown in Scheme 14.

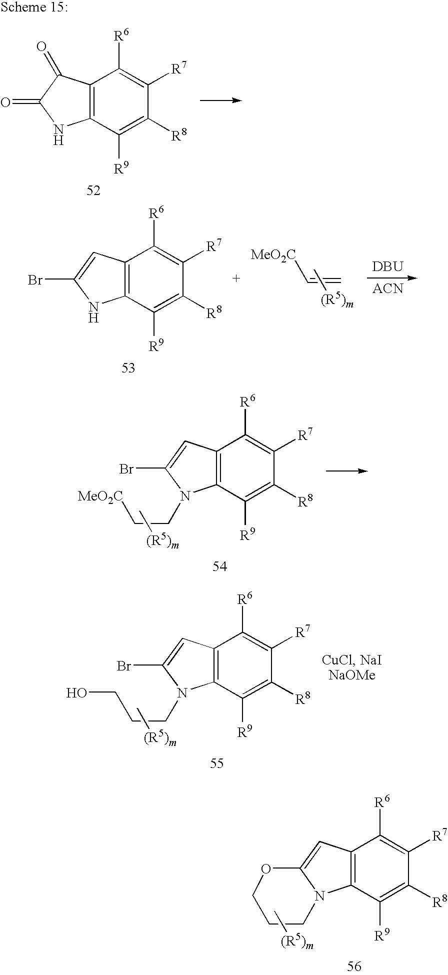

The 3,4-dihydro-2H-[1,3]oxazino[3,2-a]indole intermediate 56 could be made as shown in Scheme 15. Classical Vilsmeier-Haack formylation with POCl₃ in DMF could be used to make the tricyclic aldehyde needed to condense with ketone 1.

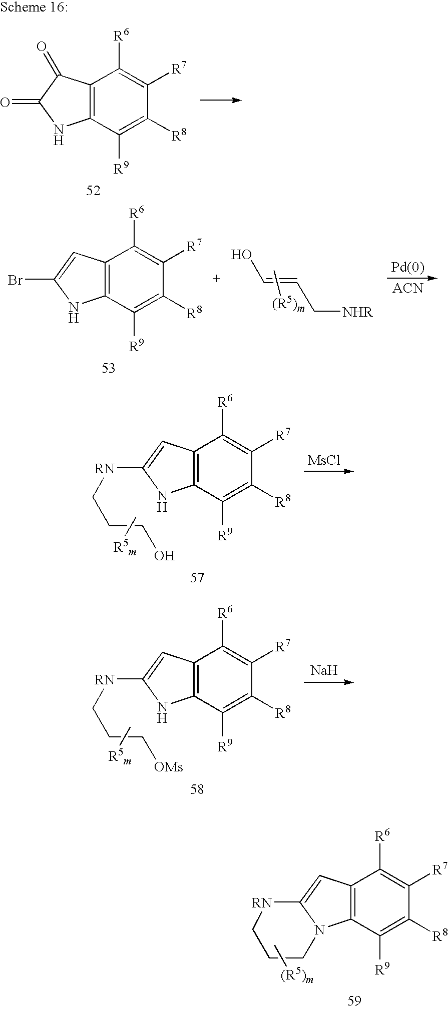

The 1,2,3,4-tetrahydropyrimido[1,2-a]indole intermediate 59 could be made as shown in Scheme 16. Classical Vilsmeier-Haack formylation with POCl₃ in DMF could be used to make the tricyclic aldehyde needed to condense with ketone 1.

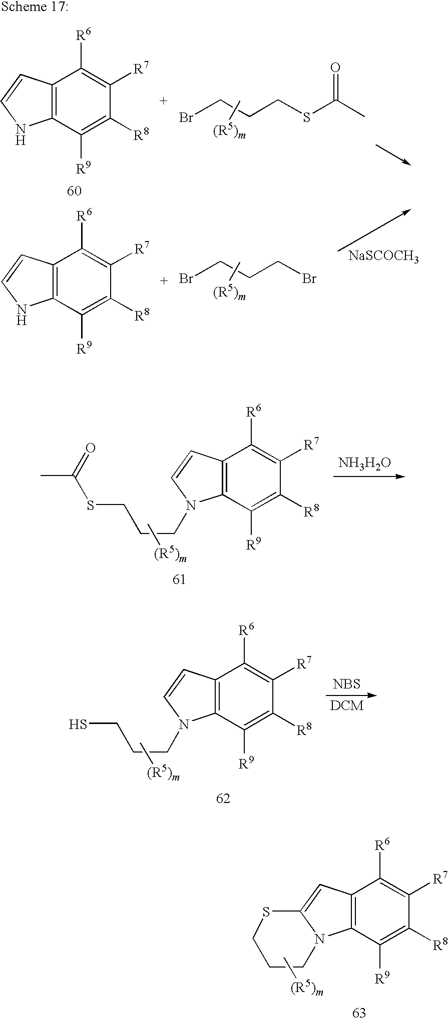

The 3,4-dihydro-2H-[1,3]thiazino[3,2-a]indole intermediate 63 could be made as shown in Scheme 17. Classical Vilsmeier-Haack formylation with POCl₃ in DMF could be used to make the tricyclic aldehyde needed to condense with ketone 1.

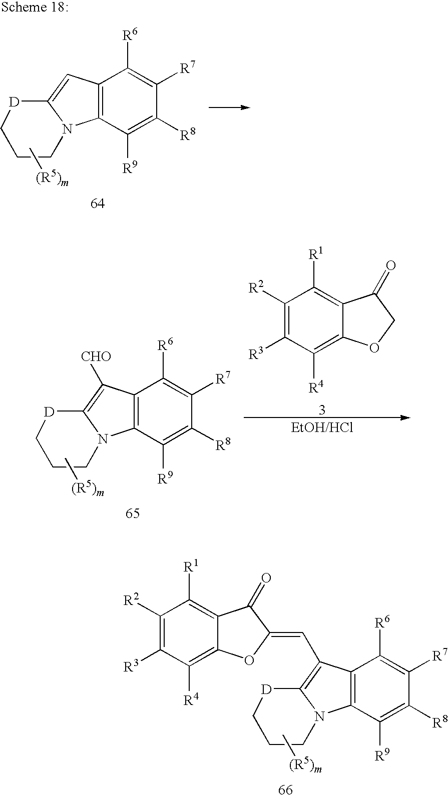

Classical Vilsmeier-Haack formylation with POCl₃ in DMF of the tricyclic intermediates 64 could be used to make the tricyclic aldehyde needed to condense with ketone 3 as shown in Scheme 18.

One of skill in the art will recognize that Schemes 1-18 can readily be adapted to produce the other [a]-fused indole compounds and pharmaceutically acceptable salts thereof according to the present invention by methods known in the art.

### EXAMPLES

The following abbreviations are used herein and have the indicated definitions: ACN is acetonitrile, AcOH is acetic acid. ATP is adenosine triphosphate. BOC is t-butoxycarbonyl. Celite™ is flux-calcined diatomaceous earth. Celite™ is a registered trademark of World Minerals Inc. CHAPS is 3[(3-cholamidopropyl)dimethylammonio]-propanesulfonic acid, DEAD is diethyl azodicarboxylate, DIAD is diisopropylazodicarboxylate, DMAP is dimethyl aminopyridine, DMF is N,N-dimethylformamide, DMF-DMA is dimethylformamide dimethyl acetal, and DMSO is dimethylsulfoxide. Dowtherm™ is a eutectic mixture of biphenyl (C₁₂H₁₀) and diphenyl oxide (C₁₂H₁₀O). Dowtherm™ is a registered trademark of Dow Corning Corporation. DPBS is Dulbecco's Phosphate Buffered Saline Formulation. EDTA is ethylenediaminetetraacetic acid, ESI stands for Electrospray Ionization, EtOAc is ethyl acetate, and EtOH is ethanol. Florisil™ is synthetic magnesia-silica gel. Florisil™ is a registered trademark of U.S. Silica Company. HEPES is 4-(2-hydroxyethyl)-1-piperazineethanesulfonic acid, GMF is Glass MicroFiber, Hunig's Base is diisopropylethylamine, HPLC is high-pressure liquid chromatography, LPS is lipopolysaccharide. Magnesol™ is a hydrated, synthetic, amorphous magnesium silicate. Magnesol™ is a registered trademark of the Dallas Group of America Inc. MeCN is also acetonitrile, MeOH is methanol, MS is mass spectrometry, and NEt₃ is triethylamine. Ni(Ra) is Raney™ nickel, a sponge-metal catalyst produced when a block of nickel-aluminum alloy is treated with concentrated sodium hydroxide. Raney™ is a registered trademark of W. R. Grace and Company. NMR is nuclear magnetic resonance, PBS is phosphate-buffered saline (pH 7.4), RPMI 1640 is a buffer (Sigma-Aldrich Corp., St. Louis, Mo., USA), SDS is dodecyl sulfate (sodium salt), SRB is Sulforhodamine B, TCA is tricholoroacetic acid, TFA is trifluoroacetic acid, THF is tetrahydrofuran, THP is tetrahydro-2H-pyran-2-yl. TLC is thin-layer chromatography, and TRIS is tris(hydroxymethyl)aminomethane.

The following Examples illustrate the synthesis of the [a]-fused indole compounds and intermediate compounds of the present invention. The scope of the invention is not limited to these Examples, but rather, encompasses the entire scope of the appended claims.

### Example A

### 4,6-dihydroxy-benzofuran-3-one (Compound 3, R¹=R³=OH)

To a solution of phloroglucinol (2 g, 16 mmol, 1 eq.) in ethyl ether (20 mL), ClCH₂CN (10 mL), ZnCl₂ (0.2 g, 1.6 mmol, 0.1 eq.) and 10% HCl/Et₂O (15 mL) were added. The mixture was stirred at room temperature overnight. The yellow precipitate (imine hydrochloride) was filtered off and washed three times with ethyl ether. Then, it was dissolved in 25 mL of water and heated at 100° C. overnight. The red solid was filtered off, washed three times with water and dried to give the title compound as a solid. Yield: 70%. MS (m/z): 167.18 (MH+).

### Example B

### Synthesis of 7-methoxy-1-oxo-3,4-dihydro-1H-2-oxa-4a-aza-fluorene-9-carbaldehyde (Compound 9)

### A. PREPARATION OF 1-(2-HYDROXY-ETHYL)-5-METHOXY-INDOLE-2-CARBOXYLIC ACID

NaH (60% dispersion in mineral oil, 0.366 g, 9.14 mmol, 2 eq.) was pre-washed with hexane and suspended in DMF (6 mL). To the resulting slurry, cooled to 0° C., a solution of ethyl 5-methoxy-indole-2-carboxylate (1 g, 4.57 mmol, 1 eq.) in DMF (8 mL) was added. The mixture was stirred at room temperature for 30 min. The reaction was cooled again to 0° C. and 2-(2-bromo-ethoxy)-tetrahydropyran (0.897 mL, 5.94 mmol, 1.3 eq.) was added. The reaction was stirred at room temperature for 48 h. DMF was evaporated and the residue was partitioned between water and EtOAc. The combined organic layers were washed with water and brine, dried on Na₂SO₄ and evaporated. The crude mixture was dissolved in EtOH (30 mL) and conc. HCl (few drops) was added. The resulting mixture was stirred at room temperature for 2 h, and then the solvent was evaporated to give crude 1-(2-hydroxy-ethyl)-5-methoxy-indole-2-carboxylic acid, which was used for the subsequent reaction without further purification. MS (m/z): 236.4 (MH+).

### B. PREPARATION OF 7-METHOXY-3,4-DIHYDRO-2-OXA-4A-AZA-FLUOREN-1-ONE

To a solution of crude 1-(2-hydroxy-ethyl)-5-methoxy-indole-2-carboxylic acid (1 g) in EtOH (24 mL), conc. HCl (1.2 mL) was added. The resulting mixture was stirred at 85° C. for 6 h, and then the solvent was evaporated. The crude mixture was purified by silica gel column chromatography (eluent: petroleum ether/EtOAc 7:3). Yield (on three steps from ethyl 5-methoxy-indole-2-carboxylate): 56%. MS (m/z): 218.16 (MH+).

### C. PREPARATION OF 7-METHOXY-1-OXO-3,4-DIHYDRO-1H-2-OXA-4A-AZA-FLUROENE-9-CARBALDEHYDE (COMPOUNDto 9)

Classical Vilsmeier-Haack procedure with POCl₃ in DMF was used. Reaction conditions: room temperature for 3 days. The crude mixture was purified by silica gel column chromatography (eluent: petroleum ether/EtOAc gradient from 9:1 to 6:4) to provide 7-methoxy-1-oxo-3,4-dihydro-1H-2-oxa-4a-aza-fluorene-9-carbaldehyde (9). Yield: 36%. MS (m/z): 246.08 (MH+).

The same procedure is used for other aldehydes 2 with D=—O— and a carbonyl (C═O) group on the 1-position of the 3,4-dihydro-1H-[1,4]oxazino[4,3-a]indole ring.

### Example C

### Synthesis of 10-formyl-8-methoxy-3,4-dihydro-1H-pyrazino[1,2-a]indole-2-carboxylic acid tert-butyl ester (Compound 16)

### A. PREPARATION OF 1-CYANOMETHYL-5-METHOXY-INDOLE-2-CARBOXYLIC ACID ETHYL ESTER

To a solution of ethyl 5-methoxy-indole-2-carboxylate (2 g, 9.13 mmol, 1 eq.) in DMF (30 mL), cooled to 0° C., NaH (60% dispersion in mineral oil, 0.438 g, 10.96 mmol, 1.2 eq.) was added. The resulting mixture was stirred at room temperature for 1 h, and then bromoacetonitrile (0.76 mL, 10.96 mmol, 1.2 eq.) was added. The mixture was stirred at room temperature for 36 h, and then the solvent was evaporated. The residue was partitioned between saturated ammonium chloride solution and EtOAc. The combined organic phase was washed with water, dried on Na₂SO₄ and evaporated to give crude 1-cyanomethyl-5-methoxy-indole-2-carboxylic acid ethyl ester, which was purified by silica gel column chromatography (eluent: petroleum ether/EtOAc 9:1). Yield: 82%. MS (m/z): 259.3 (MH+).

### B. PREPARATION OF 8-METHOXY-3,4-DIHYDRO-2H-PYRAZINO[1,2-A]INDOL-1-ONE

To a solution of 1-cyanomethyl-5-methoxy-indole-2-carboxylic acid ethyl ester (500 mg, 1.94 mmol, 1 eq.) in EtOH (20 mL), EtOAc (30 mL), 12% HCl (0.97 mL, 3.88 mmol, 2 eq.) and 10% Pd/C (192 mg) were added. The mixture was hydrogenated at 20 psi at room temperature for 6 h. The catalyst was filtered off and the solvent was evaporated. The residue was suspended in 5% K₂CO₃ solution and the resulting suspension was stirred at room temperature for 1 h. Dichloromethane was added, the layers were separated and the aqueous phase was extracted with dichloromethane. The combined organic phase was dried on Na₂SO₄ and evaporated to give crude 8-methoxy-3,4-dihydro-2H-pyrazino[1,2-a]indol-1-one, which was purified by triturating with dichloromethane. Yield: 75%. MS (m/z): 217.1 (MH+).

### C. PREPARATION OF 8-METHOXY-1,2,3,4-TETRAHYDRO-PYRAZINO[1,2-A]INDOLE

To a solution of 8-methoxy-3,4-dihydro-2H-pyrazino[1,2-a]indol-1-one (490 mg, 2.27 mmol, 1 eq.) in THF (10 mL), cooled to 0° C., LiAlH₄ (258 mg, 6.81 mmol, 3 eq.) was added in portions. The reaction mixture was stirred at 60° C. for 5 h, then was quenched at 0° C. by the addition of water (0.3 mL), 15% NaOH (0.9 mL) and again water (0.3 mL). The resulting suspension was stirred at room temperature for 1 h. The solid was removed by filtration and the filtrate was evaporated to give the pure title compound, 8-methoxy-1,2,3,4-tetrahydro-pyrazino[1,2-a]indole. Yield: 96%. MS (m/z): 203.22 (MH+).

### D. PREPARATION OF 8-METHOXY-3,4-DIHYDRO-1H-PYRAZINO[1,2-A]INDOLE-2-CARBOXYLIC ACID TERT-BUTYL ESTER

To a solution of 8-methoxy-1,2,3,4-tetrahydro-pyrazino[1,2-a]indole (390 mg, 1.93 mmol, 1 eq.) and triethylamine (403 μL, 2.89 mmol, 1.5 eq.) in dichloromethane (20 mL), cooled to 0° C., di-t-butyl dicarbonate (506 mg, 2.32 mmol, 1.2 eq.) dissolved in dichloromethane (10 mL) was added. The reaction mixture was stirred at room temperature for 2 h, then 5% NaHCO₃ was added and the aqueous phase was extracted with dichloromethane. The combined organic layers were dried on Na₂SO₄ and evaporated to give crude 8-methoxy-3,4-dihydro-1H-pyrazino[1,2-a]indole-2-carboxylic acid tert-butyl ester, which was purified by silica gel column chromatography (eluent: petroleum ether/EtOAc gradient from 9:1 to 6:4). Yield: 76%. MS (m/z): 303.2 (MH+).

### E. PREPARATION OF 10-FORMYL-8-METHOXY-3,4-DIHYDRO-1H-PYRAZINO[1,2-A]INDOLE-2-CARBOXYLIC ACID TERT-BUTYL ESTER (COMPOUND 16)

POCl₃ (227 μL, 2.48 mmol, 3 eq.) was added to DMF (2 mL) at 0° C. and the solution was stirred for 30 min. This mixture was added to a stirring solution of 8-methoxy-3,4-dihydro-1H-pyrazino[1,2-a]indole-2-carboxylic acid tert-butyl ester (250 mg, 0.828 mmol, 1 eq.) in DMF (4 mL) at 0° C. The resulting mixture was stirred at room temperature for 12 h. The reaction was poured into ice, made basic to pH 10 with saturated NaHCO₃ solution and extracted with dichloromethane. The combined organic layer was dried on Na₂SO₄ and evaporated to give a crude product that was purified by silica gel column chromatography (eluent: petroleum ether/EtOAc 7:3) to provide the desired 10-formyl-8-methoxy-3,4-dihydro-1H-pyrazino[1,2-a]indole-2-carboxylic acid tert-butyl ester 20. Yield: 77%. MS (m/z): 331.25 (MH+).

### Example D

### Synthesis of 7-methoxy-3,4-dihydro-1H-2-oxa-4a-aza-fluorene-9-carbaldehyde (Compound 22)

### A. PREPARATION OF 1-ETHOXYCARBONYLMETHYL-5-METHOXY-INDOLE-2-CARBOXYLIC ACID ETHYL ESTER

NaH (60% dispersion in mineral oil, 0.329 g, 8.22 mmol, 1.5 eq.) was added to a solution of ethyl 5-methoxy-indole-2-carboxylate (1.2 g, 5.48 mmol, 1 eq.) in DMF (15 mL), cooled to 0° C. The resulting suspension was stirred for 1 h, and then ethyl bromoacetate (0.91 mL, 8.22 mmol, 1.5 eq.) was added by drops. The ice was removed and the mixture was stirred at room temperature for 48 h. The reaction was quenched with the addition of saturated ammonium chloride solution and extracted with diethyl ether. The organic layer was washed with brine, dried on Na₂SO₄ and evaporated to give a crude product that was purified by silica gel column chromatography (eluent: petroleum ether/EtOAc 95:5) to get 1-ethoxycarbonylmethyl-5-methoxy-indole-2-carboxylic acid ethyl ester. Yield: 89%. MS (m/z): 306.2 (MH+).

### B. PREPARATION OF 2-(2-HYDROXYMETHYL-5-METHOXY-INDOL-1-YL)-ETHANOL

To a solution of 1-ethoxycarbonylmethyl-5-methoxy-indole-2-carboxylic acid ethyl ester (1.5 g, 4.92 mmol, 1 eq.) in THF (50 mL), LiAlH₄ (560 mg, 14.76 mmol, 3 eq.) was added. The reaction mixture was heated at 60° C. for 4 h, then was allowed to cool to rt. Water (0.6 mL), 15% NaOH (1.2 mL) and again water (0.6 mL) were added and the resulting suspension was stirred at room temperature for 1 h. The solid was removed by filtration and washed with MeOH. The filtrate was evaporated to give a crude product that was purified by triturating with dichloromethane/Et₂O 1:9 to provide 2-(2-hydroxymethyl-5-methoxy-indol-1-yl)-ethanol. Yield: 86%. MS (m/z): 222.20 (MH+).

### C. PREPARATION OF 7-METHOXY-3,4-DIHYDRO-1H-2-OXA-4A-AZA-FLUORENE

To a solution of 2-(2-hydroxymethyl-5-methoxy-indol-1-yl)-ethanol (150 mg, 0.678 mmol, 1 eq.) in DMF (10 mL), cooled to 0° C., NaH (60% dispersion in mineral oil, 33 mg, 0.814 mmol, 1.2 eq.) was added. The resulting suspension was stirred for 30 min, and then tosyl chloride (155 mg, 0.814 mmol, 1.2 eq.) was added. The mixture was stirred at 60° C. overnight. After cooling to room temperature, saturated ammonium chloride solution was added and the mixture was extracted with dichloromethane. The organic layer was washed with brine, dried on Na₂SO₄ and evaporated to give a mixture that was purified by silica gel column chromatography (eluent: petroleum ether/EtOAc gradient from 9:1 to 8:2) to give 7-methoxy-3,4-dihydro-1H-2-oxa-4a-aza-fluorene. Yield: 30%. MS (m/z): 204.2 (MH+).

### D. PREPARATION OF 7-METHOXY-3,4-DIHYDRO-1H-2-OXA-4A-AZA-FLUORENE-9-CARBALDEHYDE (COMPOUND 22)

Following the usual procedure for formylation, POCl₃ (175 μL, 1.91 mmol, 3 eq.) was added to DMF (3 mL) at 0° C. and the solution was stirred for 30 min. This mixture was added to a stirring solution of 7-methoxy-3,4-dihydro-1H-2-oxa-4a-aza-fluorene (130 mg, 0.637 mmol, 1 eq.) in DMF (5 mL) at 0° C. The resulting mixture was stirred at room temperature for 2 h. The reaction was poured into ice, made basic to pH 10 with 5 N NaOH, warmed to room temperature, heated at reflux for 5 min, and allowed to cool to rt. The aqueous layer was extracted with dichloromethane. The combined organic layer was dried on Na₂SO₄ and evaporated to give the pure title compound, 7-methoxy-3,4-dihydro-1H-2-oxa-4a-aza-fluorene-9-carbaldehyde 22. Yield: 58%. MS (m/z): 232.1 (MH+).

The same procedure is used for other aldehydes 2 with D=—O— and m=o.

### Example E

### Synthesis of 8-methoxy-1-oxo-1,2,3,4-tetrahydro-pyrazino[1,2-a]indole-10-carbaldehyde (Compound 24)

### A. PREPARATION OF 8-METHOXY-1-OXO-3,4-DIHYDRO-1H-PYRAZINO[1,2-A]INDOLE-2,10-DICARBALDEHYDE

Classical Vilsmeier-Haack procedure with an excess of POCl₃ in DMF was used, performing the reaction at 80° C. The crude product, 8-methoxy-1-oxo-3,4-dihydro-1H-pyrazino[1,2-a]indole-2,10-dicarbaldehyde, was used for the subsequent reaction without further purification. MS (m/z): 273.2 (MH+).

### B. PREPARATION OF 8-METHOXY-1-OXO-1,2,3,4-TETRAHYDRO-PYRAZINO[1,2-A]INDOLE-10-CARBALDEHYDE 24

Crude 8-methoxy-1-oxo-3,4-dihydro-1H-pyrazino[1,2-a]indole-2,10-dicarbaldehyde was stirred at room temperature for 3 h in 5N NaOH. Dichloromethane was added, the layers were separated and the aqueous phase was extracted with dichloromethane. The combined organic layer was dried on Na₂SO₄ and evaporated to give crude 8-methoxy-1-oxo-1,2,3,4-tetrahydro-pyrazino[1,2-a]indole-10-carbaldehyde 24, which was used without further purification, for condensation with benzofuranone compounds 7. MS (m/z): 245.1 (MH+).

The same procedure is used for the preparation other aldehydes 2 with D=N—H and a carbonyl (C═O) group on the 1-position of the 1,2,3,4-tetrahydropyrazino[1,2-a]indole ring.

### Example F

### Synthesis of 7-methoxy-2,3-dihydro-1H-pyrrolo[1,2-a]indole-9-carbaldehyde (Compound 31)

### A. PREPARATION OF ETHYL 1-(2-CHLOROETHYL)-5-METHOXY-1H-INDOLE-2-CARBOXYLATE

To a stirred solution of ethyl 5-methoxyindole-2-carboxylate (1.05 g, 5 mmol) in DMF (30 mL) at 0° C. was added sodium hydride (60% in mineral oil, 400 mg, 10 mmol). The mixture was stirred for 10 h. Then, dichloroethane (10 mL) and catalytic amount of potassium iodide were added and the mixture was stirred at room temperature for 18 h. An additional quantity of sodium hydride (60% in mineral oil, 400 mg, 10 mmol) was added, and the mixture was stirred at 60° C. for 4 h. The reaction was quenched with water and the product was extracted with ethyl acetate. The organic layer was washed with water (×2) and saturated sodium chloride solution (×2), dried over magnesium sulfate, and concentrated. The residue was purified by column chromatography over silica, eluting with hexanes to 20% ethyl acetate in hexanes. Ethyl 1-(2-chloroethyl)-5-methoxy-1H-indole-2-carboxylate (1.08 g, 77%) was obtained as a white solid. MS (m/z): 282.2 (MH+).

### B. PREPARATION OF [1-(2-CHLOROETHYL)-5-METHOXY-1H-INDOL-2-YL]METHANOL

To a stirred solution of ethyl 1-(2-chloroethyl)-5-methoxy-1H-indole-2-carboxylate (282 mg, 1 mmol) in toluene (10 mL) at −78° C. was added DIBAL (1.0 M in heptane, 5 mL, 5 mmol). The mixture was stirred for 10 min, and quenched with 1 N HCl. The mixture was filtered through a short pad of Celite™. The organic layer was separated and filtered through a short pad of Magnesol™. Concentration of the filtrate provided [1-(2-chloroethyl)-5-methoxy-1H-indol-2-yl]methanol (202 mg, 84%) as a yellow solid. MS (m/z): 240.1 (MH+).

### C. PREPARATION OF 1-(2-CHLOROETHYL)-5-METHOXY-1H-INDOLE-2-CARBALDEHYDE

A mixture of [1-(2-chloroethyl)-5-methoxy-1H-indol-2-yl]methanol (120 mg, 0.5 mmol) and activated manganese dioxide (500 mg) in dichloromethane (5 mL) was stirred at room temperature for 3 h and filtered. Concentration of the filtrate provided 1-(2-chloroethyl)-5-methoxy-1H-indole-2-carbaldehyde (110 mg, 93%) as a light tan solid. MS (m/z): 238.1 (MH+).

### D. PREPARATION OF 1-(2-CHLOROETHYL)-2-(1,3-DITHIAN-2-YL)-5-METHOXY-1H-INDOLE

To a solution of 1-(2-chloroethyl)-5-methoxy-1H-indole-2-carbaldehyde (90 mg, 0.38 mmol) and 1,3-propanedithiol (200 mg, 1.85 mmol) in dichloromethane (5 mL) was added anhydrous copper (II) sulfate (200 mg, 1.25 mol). The mixture was stirred at room temperature for 10 min and saturated sodium chloride solution was added. The organic layer was separated and filtered through a short pad of Magnesol™. Concentration of the filtrate provided 1-(2-chloroethyl)-2-(1,3-dithian-2-yl)-5-methoxy-1H-indole (83 mg, 66%) of as a white solid. MS (m/z): 328.2 (MH+).

### E. PREPARATION OF 7′-METHOXY-2′,3′-DIHYDROSPIRO[1,3-DITHIANE-2,1′-PYRROLO[1,2-A]INDOLE

To a solution of 1-(2-chloroethyl)-2-(1,3-dithian-2-yl)-5-methoxy-1H-indole (83 mg, 0.25 mmol) in THF was added catalytic amount of potassium iodide. The mixture was cooled to at −78° C. and nBuLi (2.5 M in hexanes, 0.2 mL, 0.5 mmol) was added. The mixture was stirred at −78° C. for 5 min and warmed to room temperature. Saturated sodium chloride solution was added, and the organic layer was separated and filtered through a short pad of Magnesol™. Concentration of the filtrate provided 7′-methoxy-2′,3′-dihydrospiro[1,3-dithiane-2,1′-pyrrolo[1,2-a]indole] (51 mg, 70%) as an off-white solid. MS (m/z): 292.2 (MH+).

### F. PREPARATION OF 7-METHOXY-2,3-DIHYDRO-1H-PYRROLO[1,2-A]INDOLE

To a mixture of 1.0 g of wet Raney™ nickel in 10 mL of ethanol was added 7′-methoxy-2′,3′-dihydrospiro[1,3-dithiane-2,1′-pyrrolo[1,2-a]indole] (100 mg, 0.34 mmol). The mixture was stirred at room temperature for 1 h and filtered through Celite™. Concentration led to a mixture of the desired 7-methoxy-2,3-dihydro-1H-pyrrolo[1,2-a]indole and its indoline analog (resulted from over reduction). This mixture was dissolved in 2 mL of dichloromethane, and activated manganese dioxide (200 mg) was added. The mixture was stirred at room temperature for 1 h and filtered through Magnesol™. Concentration of the filtrate provided 7-methoxy-2,3-dihydro-1H-pyrrolo[1,2-a]indole (45 mg, 70%) as a white solid. MS (m/z): 188.1 (MH+).

### G. PREPARATION OF 7-METHOXY-2,3-DIHYDRO-1H-PYRROLO[1,2-A]INDOLE-9-CARBALDEHYDE (COMPOUND 31)

Phosphorus oxychloride (305 mg, 2.0 mmol) in a flask under nitrogen was cooled to 0° C., and 2.0 mL of DMF was added in drops with stirring. The resulting mixture was stirred at 0° C. for 10 min. Then a solution of 7-methoxy-2,3-dihydro-1H-pyrrolo[1,2-a]indole (50 mg, 0.27 mmol) in 2 mL of dichloromethane was added. The mixture was stirred at 0° C. for 30 min, and quenched with water, followed by 5 drops of concentrated HCl. Ethyl acetate was added, and this two-phase mixture was stirred at 60° C. for 2 h. The organic layer was washed with saturated sodium chloride solution (×2), dried over magnesium sulfate, and filtered through Magnesol™. Concentration of the filtrate provided 7-methoxy-2,3-dihydro-1H-pyrrolo[1,2-a]indole-9-carbaldehyde 31, 23 mg, 40%) as a tan solid. MS (m/z): 216.1 (MH+).

### Example G

### Synthesis of 8-methoxy-2-methyl-1,2,3,4-tetrahydro-pyrazino[1,2-a]indole-10-carbaldehyde (Compound 33)

### A. PREPARATION OF 8-METHOXY-2-METHYL-1,2,3,4-TETRAHYDRO-PYRAZINO[1,2-A]INDOLE

To a solution of 8-methoxy-3,4-dihydro-1H-pyrazino[1,2-a]indole-2-carboxylic acid tert-butyl ester (270 mg, 0.891 mmol, 1 eq.) in THF (15 mL), cooled to 0° C., LiAlH₄ (101 mg, 2.67 mmol, 3 eq.) was added in portions. The reaction mixture was stirred at 60° C. for 4 h, then was quenched by the addition of water (0.1 mL), 15% NaOH (0.2 mL) and again water (0.1 mL). The resulting suspension was stirred at room temperature for 1 h. The solid was removed by filtration and the filtrate was evaporated to give 8-methoxy-2-methyl-1,2,3,4-tetrahydro-pyrazino[1,2-a]indole. Yield: 92%. MS (m/z): 217.22 (MH+).

### B. PREPARATION OF 8-METHOXY-2-METHYL-1,2,3,4-TETRAHYDRO-PYRAZINO[1,2-A]INDOLE-10-CARBALDEHYDE (COMPOUND 33)

Classical Vilsmeier-Haack procedure with POCl₃ in DMF was used to obtain 8-methoxy-2-methyl-1,2,3,4-tetrahydro-pyrazino[1,2-a]indole-10-carbaldehyde 33. Yield: 49%. MS (m/z): 245.2 (MH+).

### Example H

### Synthesis of 8-methoxy-2-methyl-1-oxo-1,2,3,4-tetrahydro-pyrazino[1,2-a]indole-10-carbaldehyde (Compound 39)

### A. PREPARATION OF 8-METHOXY-2-METHYL-3,4-DIHYDRO-2H-PYRAZINO[1,2-A]INDOL-1-ONE

To a solution of 8-methoxy-3,4-dihydro-2H-pyrazino[1,2-a]indol-1-one (150 mg, 0.694 mmol, 1 eq.) in DMF (5 mL), cooled to 0° C., NaH (60% dispersion in mineral oil, 33 mg, 0.833 mmol, 1.2 eq.) was added. The resulting mixture was stirred for 1 h allowing to the cooling bath to expire, then methyl iodide (52 μL, 0.833 mmol, 1.2 eq.) was added. The reaction was stirred at room temperature for 3 h, then DMF was evaporated. The residue was partitioned between water and dichloromethane. The combined organic phase was dried on Na₂SO₄ and evaporated to give 8-methoxy-2-methyl-3,4-dihydro-2H-pyrazino[1,2-a]indol-1-one. Yield: 25%. MS (m/z): 231.1 (MH+).

### B. PREPARATION OF 8-METHOXY-2-METHYL-1-OXO-1,2,3,4-TETRAHYDRO-PYRAZINO[1,2-A]INDOLE-10-CARBALDEHYDE (COMPOUND 35)

Classical Vilsmeier-Haack procedure with POCl₃ in DMF was used, performing the reaction at 80° C., to obtain 8-methoxy-2-methyl-1-oxo-1,2,3,4-tetrahydro-pyrazino[1,2-a]indole-10-carbaldehyde 35. Yield: 30%. MS (m/z): 259.2 (MH+).

The same procedure is used for other aldehydes 2 with R¹⁰=C₁-C₆alkyl and a carbonyl (C═O) group on the 1-position of the 1,2,3,4-tetrahydropyrazino[1,2-a]indole ring.

### Examples I-Q

### Condensation between 4,6-dihydroxy-benzofuran-3-one (3, R¹=R³=OH) or 6-hydroxy-benzofuran-3-one (Compound 3, R³=OH) and 5-methoxy-indole-3-carbaldehydes (Compound 2)

To a solution of the selected 5-methoxy-indole-3-carbaldehyde 2 (4 mmol, 1 eq.) and the selected benzofuran-3-one 3 (4 mmol, 1 eq.) in EtOH (16 mL), a catalytic amount of 12 N HCl was added (according to Scheme 1). The resulting mixture was stirred at 85° C. until disappearance of the starting materials and then allowed to cool to room temperature. The formed solid was recovered by filtration, washed with ethyl ether and dried under vacuum. In some cases, further purification was necessary as indicated in the Table 1, below.

### Synthesis of 1-Methyl-3-(3-oxo-2,3-dihydro-1-benzofuran-5-yl)urea (Compound 51)

### A. PREPARATION OF 2-BROMO-1-(2-HYDROXY-5-NITROPHENYL)ETHANONE (COMPOUND 48)

### METHOD 1

Into a mixture solution of nitric acid (1.5 mL) and acetic acid (10 mL) was added 2-bromo-2′-hydroxyacetophenone (1.0 g, 4.65 mmol). After 1 hr stirring at room temperature, the resulting reaction mixture was partitioned between water and ethyl acetate. The organic layer was washed with saturated NaCl aqueous solution (2×), dried over MgSO₄, filtered, concentrated, and chromatographed over a 120 g silica column, eluting with 30% ethyl acetate in hexane to provide 600.1 mg of the desired 2-bromo-1-(2-hydroxy-5-nitrophenyl)ethanone.

### METHOD 2

Fuming HNO₃ (7.5 mL) was added in drops via an additional funnel into a solution of 2-bromo-2′-hydroxyacetophenone (5.0 g, 23.25 mmol) in acetic acid (50 mL). The reaction mixture was stirred at room temperature for 15 minutes then partitioned between water and ethyl acetate. The organic layer was washed with saturated NaCl aqueous solution, dried over MgSO₄, filtered and concentrated. The residue was stirred with a solution of 30% ethyl acetate in hexane (50 mL) and suction filtered. The solid filter cake was dried further in vacuo to provide 2.84 g of pure 2-bromo-1-(2-hydroxy-5-nitrophenyl)ethanone.

### B. PREPARATION OF 5-NITRO-1-BENZOFURAN-3(2H)—ONE (COMPOUND 49)

Into a solution of 2-bromo-1-(2-hydroxy-5-nitrophenyl)ethanone (1.13 g, 4.34 mmol) in CH₃CN (20 mL) was added triethyl amine (577 μL, 4.34 mmol). The reaction mixture was stirred at room temperature for 30 minutes then concentrated. The residue was partitioned between water and ethyl acetate. The organic layer was washed with saturated NaCl aqueous solution, dried over MgSO₄, filtered, concentrated, and dried further in vacuo to provide 700.0 mg of the desired 5-nitro-1-benzofuran-3(2H)-one. MS (m/z): 178 (M−H).

### C. PREPARATION OF 5-AMINO-1-BENZOFURAN-3(2H)—ONE (COMPOUND 50)

Into a 50 mL round bottom flask 5-nitro-1-benzofuran-3(2H)-one (100 mg, 0.55 mmol), 10% Pd/C (100 mg) and dry THF (20 mL) were charged. The reaction flask was degassed 3 times using vacuum and a H₂ gas-filled balloon attached. The reaction mixture was stirred at room temperature under H₂ balloon pressure for 14 h. The resulting reaction mixture was suction filtered through a Celite™ bed, washed with THF, concentrated, and chromatographed over a 40 g silica column to provide 66.7 mg of the desired 5-amino-1-benzofuran-3(2H)-one. MS (m/z): 150.1 (M+H).

### D. PREPARATION OF 1-METHYL-3-(3-OXO-2,3-DIHYDRO-1-BENZOFURAN-5-YL)UREA (COMPOUND 51)

Into a solution of 5-amino-1-benzofuran-3(2H)-one (66.7 mg, 0.45 mmol) and triethyl amine (59.4 μL, 0.45 mmol) in CH₂Cl₂ (5 mL) was added in drops a solution of triphosgene (132.6 mg, 0.45 mmol) in CH₂Cl₂ (5 mL). After stirring 1 hour at room temperature, methylamine in THF (2N, 1.12 ml, 2.24 mmol) was added. The reaction mixture was stirred for 1 more hour, then concentrated and chromatographed over a 40 g silica column eluting with ethyl acetate to provide 61.3 mg of the desired 1-methyl-3-(3-oxo-2,3-dihydro-1-benzofuran-5-yl)urea. MS (m/z): 205.1 (M−H).

According to this procedure, the following compounds were obtained:

TABLE 1

| | | | | Yield | MS | |
|---|---|---|---|---|---|---|
| Ex. # | Compound Name | Aldehyde 2 | Benzofuranone 3 | (%) | (m/z) | Purification |
| I | 10-[(Z)-(4,6-dihydroxy-3- | 9 | R¹═R³═OH | 2 | 394.00 | Filtration |
| | oxo-1-benzofuran-2(3H)- | | | | | |
| | ylidene)methyl]-8- | | | | | |
| | methoxy-3,4-dihydro-1H- | | | | | |
| | [1,4]oxazino[4,3-a]indol-1- | | | | | |
| | one | | | | | |
| J | 10-[(Z)-(6-hydroxy-3-oxo- | 9 | R³═OH | 48 | 378.1 | Triturating with |
| | 1-benzofuran-2(3H)- | | | | | acetonitrile and |
| | ylidene)methyl]-8- | | | | | MeOH |
| | methoxy-3,4-dihydro-1H- | | | | | |
| | [1,4]oxazino[4,3-a]indol-1- | | | | | |
| | one | | | | | |
| K | (2Z)-4,6-dihydroxy-2-[(8- | 16 | R¹═R³═OH | 46 | 379.19 | Triturating with |
| | methoxy-1,2,3,4- | | | | | MeOH, |
| | tetrahydropyrazino[1,2- | | | | | dichloromethane |
| | a]indol-10-yl)methylene]- | | | | | and Et₂O |
| | 1-benzofuran-3(2H)-one | | | | | |
| L | (2Z)-6-hydroxy-2-[(8- | 16 | R³═OH | 25 | 363.19 | Preparative HPLC |
| | methoxy-1,2,3,4- | | | | | |
| | tetrahydropyrazino[1,2- | | | | | |
| | a]indol-10-yl)methylene]- | | | | | |
| | 1-benzofuran-3(2H)-one | | | | | |
| M | (2Z)-4,6-dihydroxy-2-[(8- | 22 | R³═OH | 51 | 380.05 | Filtration |
| | methoxy-3,4-dihydro-1H- | | | | | |
| | [1,4]oxazino[4,3-a]indol- | | | | | |
| | 10-yl)methylene]-1- | | | | | |
| | benzofuran-3(2H)-one | | | | | |
| N | 10-[(Z)-(4,6-dihydroxy-3- | 24 | R¹═R³═OH | 28 | 393.05 | Filtration |
| | oxo-1-benzofuran-2(3H)- | | | | | |
| | ylidene)methyl]-8- | | | | | |
| | methoxy-3,4- | | | | | |
| | dihydropyrazino[1,2- | | | | | |
| | a]indol-1(2H)-one | | | | | |
| O | (2Z)-6-hydroxy-2-[(7- | 31 | R³═OH | 33 | 348.2 | Column |
| | methoxy-2,3-dihydro-1H- | | | | | chromatography |
| | pyrrolo[1,2-a]indol-9- | | | | | |
| | yl)methylene]-1- | | | | | |
| | benzofuran-3(2H)-one | | | | | |
| P | (2Z)-4,6-dihydroxy-2-[(8- | 33 | R¹═R³═OH | 48 | 393.18 | Filtration |
| | methoxy-2-methyl-1,2,3,4- | | | | | |
| | tetrahydropyrazino[1,2- | | | | | |
| | a]indol-10-yl)methylene]- | | | | | |
| | 1-benzofuran-3(2H)-one | | | | | |
| Q | 10-[(Z)-(4,6-dihydroxy-3- | 35 | R¹═R³═OH | 19 | 407.04 | Filtration |
| | oxo-1-benzofuran-2(3H)- | | | | | |
| | ylidene)methyl]-8- | | | | | |
| | methoxy-2-methyl-3,4- | | | | | |
| | dihydropyrazino[1,2- | | | | | |
| | a]indol-1(2H)-one | | | | | |

### Biological Evaluation

mTOR Kinase Assay Methods

The routine human TOR assays with purified enzyme are performed in 96-well plates by DELFIA format as follows. Enzyme is first diluted in kinase assay buffer (10 mM HEPES (pH 7.4), 50 mM NaCl, 50 mM 13-glycerophosphate, 10 mM MnCl₂, 0.5 mM DTT, 0.25 μM microcystin LR, and 100 μg/mL BSA). To each well, 12 mL of the diluted enzyme is mixed briefly with 0.5 mL test inhibitor or the control vehicle dimethylsulfoxide (DMSO). The kinase reaction is initiated by adding 12.5 μL kinase assay buffer containing ATP and His6-S6K (substrate) to give a final reaction volume of 25 μL containing 800 ng/mL FLAG-TOR, 100 μM ATP and 1.25 μM His6-S6K. The reaction plate is incubated for 2 hours (linear at 1-6 hours) at room temperature with gentle shaking and then terminated by adding 25 μL Stop buffer (20 mM HEPES, pH 7.4), 20 mM EDTA, 20 mM EGTA). The DELFIA detection of the phosphorylated His6-S6K (Thr-389) is performed at room temperature using a monoclonal anti-P(T389)-p70S6K antibody (1A5, Cell Signaling) labeled with Europium-N1-ITC (Eu) (10.4 Eu per antibody, PerkinElmer). The DELFIA Assay buffer and Enhancement solution are purchased from PerkinElmer. The terminated kinase reaction mixture (45 μL) is transferred to a MaxiSorp plate (Nunc) containing 55 μL PBS. The His6-S6K is allowed to attach for 2 hours after which the wells are aspirated and washed once with PBS. DELFIA Assay buffer (100 μL) with 40 ng/mL Eu-P(T389)-S6K antibody is added. The antibody binding is continued for 1 hour with gentle agitation. The wells are then aspirated and washed 4 times with PBS containing 0.05% Tween-20 (PBST). DELFIA Enhancement solution (100 μL) is added to each well and the plates are read in a PerkinElmer Victor model plate reader.

### Fluorescence Polarization Assay for PI3K

### Materials

Reaction Buffer: 20 mM HEPES, pH 7.5, 2 mM MgCl₂, 0.05% CHAPS; and 0.01% BME (added fresh) Stop/Detection Buffer: 100 mM HEPES, pH 7.5, 4 mM EDTA, 0.05% CHAPS; ATP 20 mM in water; PIP2 (diC8, Echelon, Salt Lake City Utah, cat# P-4508) 1 mM in water (MW=856.5); GST-GRP 1.75 mg/mL or 1.4 mg/mL in 10% glycerol; Red detector (TAMRA) 2.5 μM; Plate: Nunc 384 well black polypropylene fluorescence plate.

### Methods

PI3-Kinase reactions were performed in 5 μM HEPES, pH 7, 2.5 μM MgCl₂, and 25 μM ATP, with diC8-PI(4,5)P2 (Echelon, Salt Lake City Utah) as substrate. Nunc 384 well black polypropylene fluorescent plates were used for PI3K assays. Reactions were quenched by the addition of EDTA to a final concentration of 10 μM. Final reaction volumes were 10 μl. For evaluation of PI3K inhibitors, 5 ng of enzyme (PI3K-alpha, beta, gamma, or delta) and 2.5 μM of substrate was used per 10 ml reaction volume, and inhibitor concentrations ranged from 100 pM to 20 μM; the final level of DMSO in reactions never exceeded 2%. Reactions were allowed to proceed for one hour at 25° C. After 1 hour, GST-tagged GRP1 (general receptor for phosphoinositides) PH domain fusion protein was added to a final concentration of 100 nM, and BODIPY-TMRI(1,3,4,5)P4 (Echelon) was also added to a final concentration of 5 nM. Final sample volumes were 25 μl with a final DMSO concentration of 0.8%. Assay Plates were read on PerkinElmer Envision plate readers with appropriate filters for Tamra [BODIPY-TMRI(1,3,4,5)P4]. Data obtained were used to calculate enzymatic activity and enzyme inhibition by inhibitor compounds.

### In Vitro Cell Culture Growth Assay Methods

Cell lines used were human adenocarcinoma (LoVo), pancreatic (PC3), prostate (LNCap), breast (MDA468, MCF7), colon (HCT116), renal (HTB44 A498), and ovarian (OVCAR3) tumor cell lines. The tumor cells were plated in 96-well culture plates at approximately 3000 cells per well. One day following plating, various concentrations of PI3K inhibitors in DMSO were added to cells (final DMSO concentration in cell assays was 0.25%). Three days after drug treatment, viable cell densities were determined by cell mediated metabolic conversion of the dye MTS, a well-established indicator of cell proliferation in vitro. Cell growth assays were performed using kits purchased from Promega Corporation (Madison, Wis.), following the protocol provided by the vendor. Measuring absorbance at 490 nm generated the MTS assay results. Compound effect on cell proliferation was assessed relative to untreated control cell growth. The drug concentration that conferred 50% inhibition of growth was determined as IC₅₀ (μM).

Table 2 shows the results of the described biological assays.

TABLE 2

| | TOR | PI3 Kinase | PI3 Kinase | | |
|---|---|---|---|---|---|
| | Kinase | α | γ | OVCAR3 | PC3-MM2 |
| Example | IC₅₀ (μM) | IC₅₀ (nM) | IC₅₀ (nM) | IC₅₀ (μM) | IC₅₀ (μM) |
| I | 0.024 | 1.1 | 9.0 | | 5.63 |
| J | 0.114 | 21.5 | 76.5 | 5.28 | >10.00 |
| K | 0.009 | 5.5 | 115.5 | 1.62 | 2.19 |
| L | 0.320 | 16.5 | 115.5 | 0.78 | 1.51 |
| M | <0.032 | 4.5 | 57.0 | 1.00 | 1.91 |
| N | <0.032 | 2.5 | 24.5 | 4.95 | >10.00 |
| 0 | 0.066 | 14.0 | 76.0 | 3.72 | 3.40 |
| P | 0.009 | 6.0 | 30.5 | 1.67 | 1.61 |
| Q | 0.004 | 3.0 | 27.0 | 3.60 | 5.05 |

While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

Throughout this application, various publications are referenced. The disclosures of these publications in their entireties are hereby incorporated by reference into this application in order to more fully describe the state of the art as known to those skilled therein as of the date of the invention described and claimed herein.

What is claimed is:

1. A compound of the Formula I: 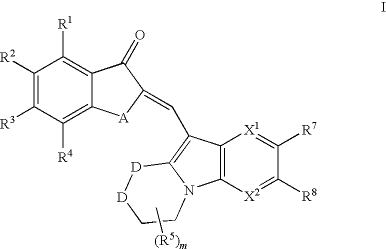
or a pharmaceutically acceptable salt thereof, wherein
A is —O— or —S—;
X¹ is N or C—R⁶;
X² is N or C—R⁹;
with the proviso that at most one of X¹ and X² can be N;
R¹ is H, C₁-C₆alkyl, hydroxyl, C₁-C₆alkyl-NHC(O)NH—, C₂-C₁₀alkenyl-NHC(O)NH—, C₂-C₁₀alkynyl-NHC(O)NH—, C₁-C₆hydroxylalkyl-NHC(O)NH—, amino(C₁-C₆alkyl)-NHC(O)NH—, or C₁-C₆alkoxy;
R² is H, C₁-C₆alkyl, hydroxyl, C₁-C₆alkyl-NHC(O)NH—, C₂-C₁₀alkenyl-NHC(O)NH—, C₂-C₁₀alkynyl-NHC(O)NH—, C₁-C₆hydroxylalkyl-NHC(O)NH—, amino(C₁-C₆alkyl)-NHC(O)NH—, or C₁-C₆alkoxy;
R³ is H, C₁-C₆alkyl, hydroxyl, C₁-C₆alkyl-NHC(O)NH—, C₂-C₁₀alkenyl-NHC(O)NH—, C₂-C₁₀alkynyl-NHC(O)NH—, C₁-C₆hydroxylalkyl-NHC(O)NH—, amino(C₁-C₆alkyl)-NHC(O)NH—, or C₁-C₆alkoxy;
R⁴ is H, C₁-C₆alkyl, hydroxyl, C₁-C₆alkyl-NHC(O)NH—, C₂-C₁₀alkenyl-NHC(O)NH—, C₂-C₁₀alkynyl-NHC(O)NH—, C₁-C₆hydroxylalkyl-NHC(O)NH—, amino(C₁-C₆alkyl)-NHC(O)NH—, or C₁-C₆alkoxy;
R⁶ is H, C₁-C₆alkyl, hydroxyl, or C₁-C₆alkoxy;
R⁷ is H, C₁-C₆alkyl, hydroxyl, or C₁-C₆alkoxy;
R⁸ is H, C₁-C₆alkyl, hydroxyl, or C₁-C₆alkoxy;
R⁹ is H, C₁-C₆alkyl, hydroxyl, or C₁-C₆alkoxy;
one of D is —O—, —N(R¹⁰)—, (CH₂)ₙ, or —S(O)ₒ— and the other D is CH₂;
m is 0, 1, 2, or 3;
n, and o are independently 0, 1, or 2;
R¹⁰ is H, (C₁-C₆alkoxy)carbonyl, C₁-C₆alkyl, (C₁-C₆alkyl)amido, C₁-C₉heterocycle, C₃-C₈cycloalkyl, or C₆-C₁₄aryl;
R⁵ are independently C₁-C₆alkyl, hydroxyl, or C₁-C₆alkoxy;
or two R⁵ groups on the same carbon atom, when taken together with the carbon to which they are attached, can form a carbonyl (C═O) group.

2. A compound of the Formula II: 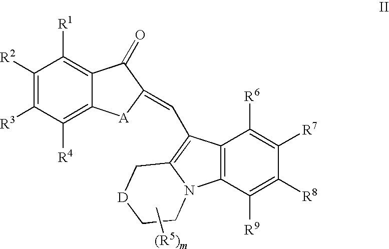
or a pharmaceutically acceptable salt thereof, wherein
A is —O— or —S—;
R¹ is H, C₁-C₆alkyl, hydroxyl, C₁-C₆alkyl-NHC(O)NH—, C₂-C₁₀alkenyl-NHC(O)NH—, C₂-C₁₀alkynyl-NHC(O)NH—, C₁-C₆hydroxylalkyl-NHC(O)NH—, amino(C₁-C₆alkyl)-NHC(O)NH—, or C₁-C₆alkoxy;
R² is H, C₁-C₆alkyl, hydroxyl, C₁-C₆alkyl-NHC(O)NH—, C₂-C₁₀alkenyl-NHC(O)NH—, C₂-C₁₀alkynyl-NHC(O)NH—, C₁-C₆hydroxylalkyl-NHC(O)NH—, amino(C₁-C₆alkyl)-NHC(O)NH—, or C₁-C₆alkoxy;
R³ is H, C₁-C₆alkyl, hydroxyl, C₁-C₆alkyl-NHC(O)NH—, C₂-C₁₀alkenyl-NHC(O)NH—, C₂-C₁₀alkynyl-NHC(O)NH—, C₁-C₆hydroxylalkyl-NHC(O)NH—, amino(C₁-C₆alkyl)-NHC(O)NH—, or C₁-C₆alkoxy;
R⁴ is H, C₁-C₆alkyl, hydroxyl, C₁-C₆alkyl-NHC(O)NH—, C₂-C₁₀alkenyl-NHC(O)NH—, C₂-C₁₀alkynyl-NHC(O)NH—, C₁-C₆hydroxylalkyl-NHC(O)NH—, amino(C₁-C₆alkyl)-NHC(O)NH—, or C₁-C₆alkoxy;
R⁶ is H, C₁-C₆alkyl, hydroxyl, or C₁-C₆alkoxy;
R⁷ is H, C₁-C₆alkyl, hydroxyl, or C₁-C₆alkoxy;
R⁸ is H, C₁-C₆alkyl, hydroxyl, or C₁-C₆alkoxy;
R⁹ is H, C₁-C₆alkyl, hydroxyl, or C₁-C₆alkoxy;
D is —O—, —N(R¹⁰)—, (CH₂)ₙ, or —S(O)ₙ—;
m is 0, 1, 2, or 3;
n, and o are independently 0, 1, or 2;
R¹⁰ is H, (C₁-C₆alkoxy)carbonyl, C₁-C₆alkyl, (C₁-C₆alkyl)amido, C₁-C₉heterocycle, C₃-C₈cycloalkyl, or C₆-C₁₄aryl;
R⁵ are independently C₁-C₆alkyl, hydroxyl, or C₁-C₆alkoxy;
or two R⁵ groups on the same carbon atom, when taken together with the carbon to which they are attached, can form a carbonyl (C═O) group.

3. The compound of claim 2, wherein A is —O—.

4. The compound of claim 2, wherein R¹ is H or hydroxyl.

5. The compound of claim 2, wherein R² is H.

6. The compound of claim 2, wherein R² is C₁-C₆alkyl-NHC(O)NH—.

7. The compound of claim 2, wherein R³ is hydroxyl.

8. The compound of claim 2, wherein R⁴ is H.

9. The compound of claim 2, wherein R⁶ is H.

10. The compound of claim 2, wherein R⁷ is C₁-C₆alkoxy.

11. The compound of claim 10, wherein R⁷ is methoxy.

12. The compound of claim 2, wherein R⁸ is H.

13. The compound of claim 2, wherein R⁹ is H.

14. The compound of claim 2, wherein A is —O—, R¹ is H or hydroxyl, R² is H, R³ is hydroxyl, R⁴ is H, R⁶ is H, R⁷ is methoxy, R⁸ is H, and R⁹ is H.

15. The compound of claim 2, wherein D is —O—.

16. A compound selected from the group consisting of:
10-[(Z)-(4,6-dihydroxy-3-oxo-1-benzofuran-2(3H)-ylidene)methyl]-8-methoxy-3,4-dihydro-1H-[1,4]oxazino[4,3-a]indol-1-one;
10-[(Z)-(6-hydroxy-3-oxo-1-benzofuran-2(3H)-ylidene)methyl]-8-methoxy-3,4-dihydro-1H-[1,4]oxazino[4,3-a]indol-1-one;
and (2Z)-4,6-dihydroxy-2-[(8-methoxy-3,4-dihydro-1H-[1,4]oxazino[4,3-a]indol-10-yl)methylene]-1-benzofuran-3(2H)-one.

17. The compound of claim 2, wherein D is NR¹⁰.

18. A compound selected from the group consisting of:
(2Z)-4,6-dihydroxy-2-[(8-methoxy-1,2,3,4-tetrahydropyrazino[1,2-a]indol-10-yl)methylene]-1-benzofuran-3(2H)-one;
(2Z)-6-hydroxy-2-[(8-methoxy-1,2,3,4-tetrahydropyrazino[1,2-a]indol-10-yl)methylene]-1-benzofuran-3(2H)-one;
10-[(Z)-(4,6-dihydroxy-3-oxo-1-benzofuran-2(3H)-ylidene)methyl]-8-methoxy-3,4-dihydropyrazino[1,2-a]indol-1(2H)-one;
(2Z)-4,6-dihydroxy-2-[(8-methoxy-2-methyl-1,2,3,4-tetrahydropyrazino[1,2-a]indol-10-yl)methylene]-1-benzofuran-3(2H)-one;
and 10-[(Z)-(4,6-dihydroxy-3-oxo-1-benzofuran-2(3H)-ylidene)methyl]-8-methoxy-2-methyl-3,4-dihydropyrazino[1,2-a]indol-1(2H)-one.

19. The compound of claim 2, wherein D is (CH₂)ₙ.

20. The compound (2Z)-6-hydroxy-2-[(7-methoxy-2,3-dihydro-1H-pyrrolo[1,2-a]indol-9-yl)methylene]-1-benzofuran-3(2H)-one.

21. A composition comprising the compound of claim 1, and a pharmaceutically acceptable carrier.

22. The composition of claim 21, wherein the pharmaceutically acceptable carrier is suitable for oral administration and the composition comprises an oral dosage form.

23. A composition comprising a compound of claim 1; a second compound selected from the group consisting of a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, and lavendustin A; and a pharmaceutically acceptable carrier.

24. The composition of claim 23, wherein the second compound is Avastin.

25. A method of inhibiting PI3K, comprising administering to a mammal the compound of claim 1 in an amount effective to inhibit PI3K.

26. A method of inhibiting mTOR, comprising administering to a mammal the compound of claim 1 in an amount effective to inhibit mTOR.

27. A method of treating advanced renal cell carcinoma, comprising administering to a mammal in need thereof the compound of claim 1 in an amount effective to treat advanced renal cell carcinoma.

28. A method of treating acute lymphoblastic leukemia, comprising administering to a mammal in need thereof the compound of claim 1 in an amount effective to treat acute lymphoblastic leukemia.

29. A method of treating malignant melanoma, comprising administering to a mammal in need thereof the compound of claim 1 in an amount effective to treat malignant melanoma.

30. A method of treating soft-tissue or bone sarcoma, comprising administering to a mammal in need thereof the compound of claim 1 in an amount effective to treat soft-tissue or bone sarcoma.

31. A method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof the composition of claim 24 in an amount effective to treat the cancer.

32. A method of synthesizing a compound of claim 2 comprising reacting the keto heterocycle: 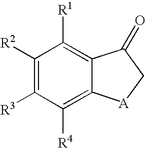
with an [a]-fused indole aldehyde: 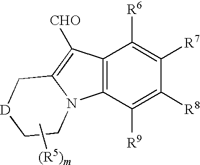
wherein R¹-R⁹, A, D, and m are as defined in claim 2, to give the [a]-fused indole II: 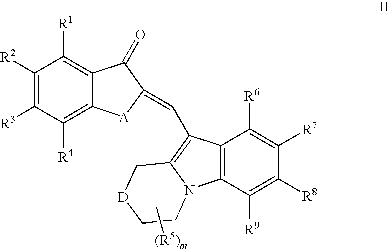
or a pharmaceutically acceptable salt thereof.

33. The method of claim 32 further comprising reacting the tricyclic intermediate: 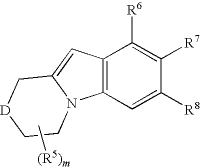
with POCl₃ and DMF thereby producing the aldehyde: 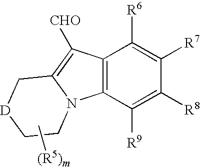
by formylating the free position on the indole ring.

34. The method of claim 33 when D is O or S(O)ₒ, wherein o is 0, 1, or 2, further comprising:
a. reacting the indole ester: 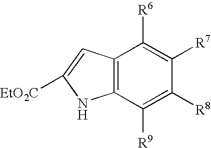
with the alkylating agent shown, where X is halogen: 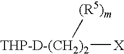
b. removal of the protecting group;
c. ring closure to produce: 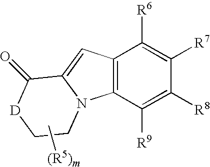
d. reacting lactone or thiolactone with DIBAL producing an intermediate 3,4-dihydro-1H-[1,4]oxazino[4,3-a]indol-1-ol or 3,4-dihydro-1H-[1,4]thiazino[4,3-a]indol-1-ol;
e. reacting the hemiacetal produced with a trialkylsilyl hydride: 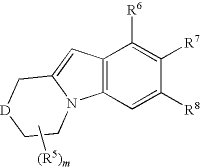
producing the intermediate lacking a carbonyl group.

35. The method of claim 33 when D is N(R¹⁰), wherein R¹⁰ is H, (C₁-C₆alkoxy)carbonyl, C₁-C₆alkyl, (C₁-C₆alkyl)amido, C₁-C₉heterocycle, C₃-C₈cycloalkyl, or C₆-C₁₄aryl, further comprising:
(a) reacting the indole ester with the alkylating agent shown where X is halogen; 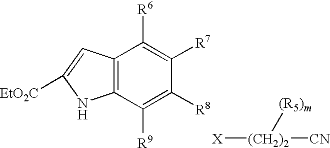
to replace the hydrogen atom on the nitrogen atom at position 1 of the indole ring;
(b) reduction and cyclization of the nitrile intermediate producing the lactam: 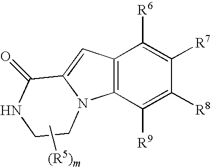
(c) optionally reacting the lactam with alkylating agent R¹⁰—X, where X is halogen, producing an intermediate lactam: 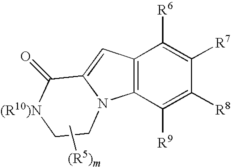
(d) reducing the lactam with LAH producing the intermediate: 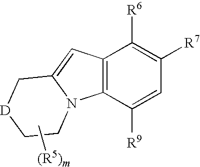
thereby removing the oxygen atom from the carbonyl group.

36. The method of claim 33 when D is (CH₂), and n is 0, 1, or 2, further comprising:
(a) reacting the indole ester with the alkylating agent shown where X is halogen; 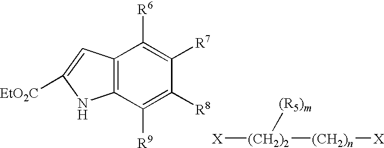
thereby producing: 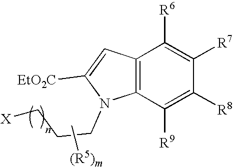
(b) reducing the ester with DIBAL, producing an intermediate allylic alcohol;
(c) oxidizing the alcohol with MnO₂ to make an aldehyde;
(d) condensing the aldehyde with propane-1,3-dithiol producing the 1,3-dithiane: 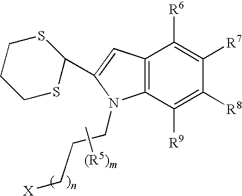
affecting ring closure under basic conditions producing the tricyclic intermediate: 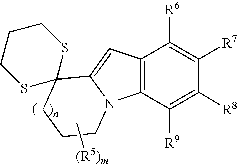
(e) removing the dithiane masking group thereby making tricyclic intermediate: 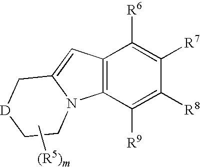
lacking a carbonyl group.
